(19) United States
(12) Patent Application Publication
(10) Pub. No.: US 2012/0024382 A1
(43) Pub. Date: Feb. 2, 2012

(54) NOVEL COMPOUNDS, DERIVATIVES THEREOF AND THEIR USE IN HETEROJUNCTION DEVICES

(75) Inventors: Andrew Holmes, Carlton (AU); David Jones, Carlton (AU); Wing Ho Wallace Wong, Carlton (AU); Chang Qi Ma, Ulm (DE); Peter Baeuerle, Elchingen (DE)

(73) Assignees: THE UNIVERSITY OF ULM, ULM (DE); THE UNIVERSITY OF MELBOURNE, PARKVILLE (AU)

(21) Appl. No.: 13/131,598

(22) PCT Filed: Nov. 27, 2009
(86) PCT No.: PCT/AU09/01563

§ 371 Date: Sep. 21, 2011

(30) Foreign Application Priority Data
Nov. 28, 2008 (AU) 2008906181

(51) Int. Cl. H01L 51/46; C07C 271/28; C07F 7/08; C07C 25/22; B82Y 99/00

(52) U.S. Cl. 136/263; 556/432; 570/183; 556/424; 560/27; 556/488; 570/129; 977/734; 977/948

(57) ABSTRACT

The invention relates to novel polyaromatic and polyheteroaromatic compounds and derivatives thereof. The compounds display high solubility in organic solvents. A further aspect of the invention relates to the use of the novel compounds in the fabrication of organic film based heterojunction devices. In one form the devices display high conversion efficiencies in solar cell applications.

### FIELD OF INVENTION

The present invention relates to novel polyaromatic and polyheteroaromatic compounds and derivatives thereof and their use in the fabrication of organic film based heterojunction devices. In one form the devices display high conversion efficiencies in solar cell applications.

### BACKGROUND

Solid state heterojunctions such as the pn junction between p-type and n-type semiconductors have found widespread application in modern electronics.

Organic film based organic photovoltaic (OPV) materials are potentially a competitive alternative to silicon, offering advantages in flexibility, large-scale manufacture by reel-to-reel printing technology, low cost, large area and ease of installation. Organic devices consist of bulk-heterojunction cells that may be fabricated using either conjugated small molecule-fullerene blends, conjugated polymer-fullerene blends or polymer-polymer blends. The standard way of assessing device performance is the efficiency with which solar energy is converted into electrical energy (% ece) which depends on the product of the open circuit voltage (V_{oc}), the short circuit current (J_{sc}) and the fill factor (FF) divided by the input power per unit area [“Organic Photovoltaics”, Brabec, C.; Dyakonov, V.; Scherf, U. (Eds.), Wiley-VCH, Weinheim 2008 ISBN: 978-3-527-31675-5; Gregg, B. A. MRS Bull. 2005, 30, 20-22].

Small molecule-fullerene heterojunction solar cells have been fabricated from blends of electron rich donor (Don) molecules with electron deficient acceptor (Acc) solution-processible fullerene or perylene diimide derivatives [Schmidt-Mende, L.; Fechtenkötter, A.; Müllen, K.; Moons, E.; Friend, R. H.; MacKenzie, J. D. Science 2001, 293, 1119-1122; Tamayo, A. B.; Tantiwiwat, M.; Walker, B.; Nguyen, T.-Q. J. Phys. Chem. C 2008, 112, 15543-15552; Ma, C.-Q.; Mena-Osteritz, E.; Debaerdemaeker, T.; Wienk, M. M.; Janssen, R. A.; Baeuerle, P. Angew. Chem. Int. Ed. 2007, 46, 1679-1683; Ma, C.-Q.; Fonrodona, M.; Schikora, M. C.; Wienk, M. M.; Janssen, R. A.; Baeuerle, P. Adv. Funct. Mater. 2008, 18, 3323-3331]. The open circuit voltage is determined by the difference in the energy between the Highest Occupied Molecular Orbital (HOMO) of the donor molecule and the Lowest Unoccupied Molecular Orbital (LUMO) of the acceptor molecule.

Hexabenzocoronene (HBC) is a planar aromatic molecule consisting of thirteen fused six membered rings [Wu, J.; Pisula, W.; Müllen, K. Chem. Rev. 2007, 107, 718-747]. HBCs belong to the family of polycyclic aromatic hydrocarbons consisting of flat disc-like cores. HBC and its derivatives have been shown to self assemble into columnar structures giving rise to ordered morphology in films [Ito, S.; Wehmeier, M.; Brand, J. D.; Kubel, C.; Epsch, R.; Rabe, J. P.; Müllen, K. Chem. Eur. J. 2000, 6, 4327-4342; Kastler, M.; Pisula, W.; Wasserfallen, D.; Pakula, T.; Müllen, K. J. Am. Chem. Soc. 2005, 127, 4286-4296]. This property is potentially very useful in bulk heterojunction solar cells where the active layer consists of an electron and a hole transport material usually blended together in a random fashion [Sergeyev, S.; Pisula, W.; Geerts, Y. H. Chem. Soc. Rev. 2007, 36, 1902-1929; Simpson, C. D.; Wu, J.; Watson, M. D.; Müllen, K. J. Mater. Chem. 2004, 14, 494-504]. The self assembly of materials into ordered structures in a bulk heterojunction increases the efficiency of the photovoltaic device by facilitating charge separation and transport.

The chemistry of the core structure of HBC has been established by the group of Müllen in the last decade [Wu, J.; Pisula, W.; Müllen, K. Chem. Rev. 2007, 107, 718-747]. Many HBC derivatives with alkyl substituents have been reported. Some derivatives have been shown to π-π stack in solid state by x-ray crystallography [Wu, J.; Grimsdale, A. C.; Müllen, K. J. Mater. Chem. 2005, 15, 41-52] while others were identified by atomic force microscopy (AFM) imaging and a variety of spectroscopic techniques to assemble into columnar structures [Kastler, M.; Pisula, W.; Wasserfallen, D.; Pakula, T.; Müllen, K. J. Am. Chem. Soc. 2005, 127, 4286-4296; Ito, S.; Wehmeier, M.; Brand, J. D.; Kubel, C.; Epsch, R.; Rabe, J. P.; Müllen, K. Chem. Eur. J. 2000, 6, 4327-4342]. Extended HBC derivatives have also been synthesised and graphitic sheets of over 400 carbon atoms have been isolated and identified [Simpson, C. D.; Mattersteig, G.; Martin, K.; Gherghel, L.; Bauer, R. E.; Räder, H. J.; Müllen, K. J. Am. Chem. Soc. 2004, 126, 3139-3147]. Solution processibility has only been achieved by the introduction of long chain alkyl or amphiphilic substituents at the terminus of the peripheral conjugated units.

Organic solar cell devices have been fabricated using HBC derivatives [Schmidt-Mende, L.; Fechtenkotter, A.; Müllen, K.; Moons, E.; Friend, R. H.; MacKenzie, J. D. Science 2001, 293, 1119-1122; Schmidt-Mende, L.; Watson, M.; Müllen, K.; Friend, R. H. Mol. Cryst. Liq. Cryst. 2003, 396, 73-90; Hassheider, T.; Benning, S. A.; Lauhof, M. W.; Kitzerow, H. S.; Bock, H.; Watson, M. D.; Muellen, K. Mol. Cryst. Liq. Cryst. 2004, 413, 2597-2608; Jung, J.; Rybak, A.; Slazak, A.; Bialecki, S.; Miskiewicz, P.; Glowacki, I.; Ulanski, J.; Rosselli, S.; Yasuda, A.; Nelles, G.; Tomovic, Z.; Watson, M. D.; Muellen, K. Synth. Met. 2005, 155, 150-156; Schmidtke, J. P.; Friend, R. H.; Kastler, M.; Müllen, K. J. Chem. Phys. 2006, 124, 174704/1-174704/6; Li, J.; Kastler, M.; Pisula, W.; Robertson, J. W. F.; Wasserfallen, D.; Grimsdale, A. C.; Wu, J.; Müllen, K. Adv. Funct. Mater. 2007, 17, 2528-2533]. In all cases, the HBC derivatives were used in conjunction with perylene diimide in bulk heterojunction devices with a general structure of ITO (indium tin oxide)/PEDOT (poly(3,4-ethylenedioxythiophene):PSS (polystyrenesulfonate)/HBC-perylene diimide blend/Al. Power conversion efficiency measured over the entire solar spectrum was not reported. To date, the results of solution processed HBCs in organic photovoltaic devices have not been promising.

The group of Aida has reported an amphiphilic HBC system which has been shown to assemble into nanotube structures [Hill, J. P.; Jin, W.; Kosaka, A.; Fukushima, T.; Ichihara, H.; Shimomura, T.; Ito, K.; Hashizume, T.; Ishii, N.; Aida, T. Science 2004, 304, 1481-1483]. These amphiphilic HBC derivatives have been fabricated into macroscopic fibers [Yamamoto, Y.; Fukushima, T.; Jin, W.; Kosaka, A.; Hara, T.; Nakamura, T.; Saeki, A.; Seki, S.; Tagawa, S.; Aida, T. Adv. Mater. 2006, 18, 1297-1300], chiral nanocoils [Yamamoto, T.; Fukushima, T.; Kosaka, A.; Jin, W.; Yamamoto, Y.; Ishii, N.; Aida, T. Angew. Chem. Int. Ed. 2008, 47, 1672-1675] and photoconducting donor-acceptor heterojunction assemblies [Yamamoto, Y.; Fukushima, T.; Saeki, A.; Seki, S.; Tagawa, S.; Ishii, N.; Aida, T. J. Am. Chem. Soc. 2007, 129, 9276-9277]. To date, amphiphilic HBCs have not been suitable for fabrication in organic solar cells.

HBC derivatives have been described in use in electrical or optical components [Watson, M. D.; Müllen, K. 2004, DE10255363, 12 pp, CAN 141:45809] and in photoconductive nanotubes [Yamamoto, Y.; Fukushima, T.; Isago, Y.; Ogawa, A.; Aida, T. 2007, JP2007238544, 20 pp, CAN 147:374056.].

Coronene charge-transport materials, methods of fabrication thereof, and methods of use thereof have been reported [Marder, S.; Zesheng, A.; Yu, J.; Kippelen, B. 2006, WO2006093965, 90 pp, CAN 145:326126]. The use of hexabenzocoronenes in hydrogen storage [Pez, G. P.; Scott, A. R.; Cooper, A. C.; Cheng, H.; Bagzis, L. D.; Appleby, J. B. 2005, WO2005000457, 133 pp, CAN 142:117630] and in sensor applications [Nuckolls, C.; Guo, X.; Kim, P.; Xiao, S.; Myers, M. 2007, WO2007133288, 48 pp, CAN 148:4383] have been disclosed. The use of planar organic compounds in organic light emitting [Samuel, I. D. W.; Halim, M.; Burn, P. L.; Pillow, J. N. G. 1999, WO9921935, 71 pp, CAN 130:330417] and organic field effect transistor devices [Nanpo, H. 2005, JP2005079163, 8 pp, CAN 142:308143] has also been disclosed.

In the fabrication of devices on a large area with low cost components, solution processible molecules, that is molecules that have sufficient solubility in organic solvents, are ideal, especially those that form good amorphous films. There is a significant advantage over vacuum deposition in the reduction in the complexity of steps and the ability to fabricate large area devices.

Accordingly, it would be desirable to provide molecules that have good solubility in solvents, are capable of self organisation and that are flexible in design so as to provide control over the molecules electronic energy levels and increase charge transport mobilities. Such molecules would find advantageous application in organic heterojunction devices.

### SUMMARY OF INVENTION

In a first aspect of the invention there is provided a conjugated compound comprising a conjugated linear or branched polycyclic aromatic or heteroaromatic core, said core being peripherally substituted with at least one conjugated aromatic or heteroaromatic moiety, said moiety or moieties comprising at least one substituent conferring solubility on said compound. Preferably, the conjugated aromatic or heteroaromatic moiety or moieties modify charge transport mobility within said compound. Preferably, the solubility conferring substituents confer solubility of said compound in an organic solvent.

In a preferred embodiment of the first aspect of the invention the conjugated aromatic or heteroaromatic moiety or moieties further comprise at least one terminal substituent located at the conjugation terminus or termini of said moiety or moieties said terminal substituent having reactive functionality.

In a further preferred embodiment of the first aspect of the invention, the core preferably comprises at least three fused or linked aromatic or heteroaromatic rings. Suitable cores may be selected from linear or branched polycyclic aromatics, polycyclic aromatics containing heteroatoms, such as, for example, nitrogen, oxygen, sulphur, phosphorous, boron, silicon or germanium, porphyrins, confused porphyrins, porphyrazines, phthalothocyanines, and their metal containing analogues.

In a particularly preferred embodiment, the core is a hexabenzocoronene.

The solubility conferring substituents may be one or more branched or unbranched, linear or cyclic, substituted or unsubstituted hydrocarbyl groups or, alternatively or additionally, groups that confer amphiphilic character on the whole molecule. The hydrocarbyl groups may be substituted with a variety of substituents comprising linear, branched or cyclic and/or heteroatom containing substituents. Preferably, the solubility conferring substituent is a branched or unbranched, substituted or unsubstituted, linear or cyclic alkyl, alkenyl, or alkynyl group, especially a long chain alkyl, alkenyl or alkynyl group having from between 4 and 30 carbon atoms.

More preferably, the long chain alkyl group has from between 6 and 20 carbon atoms.

Particularly preferred solubility conferring substituents are branched or unbranched, substituted or unsubstituted, cyclic or linear alkyl, alkenyl, or alkynyl groups, for example, n-hexyl, n-heptyl, n-octyl, n-nonyl, n-decyl, n-dodecyl, n-hexenyl, n-octenyl, n-decenyl, n-hexynyl, n-octynyl, n-decynyl and branched isomers thereof.

Advantageously, the solubility conferring substituents may be laterally placed on the conjugated aromatic or heteroaromatic moiety or moieties. By laterally placed it is meant that the solubility conferring substituent(s) is/are not present on the conjugation terminus or termini of the conjugated aromatic or heteroaromatic moiety or moieties.

The substituent having reactive functionality may be any substituent that is capable of forming, through suitable reaction, a carbon-carbon bond or a carbon-heteroatom bond. A preferred substituent comprises a halo, alkenyl, alkynyl, aldehyde, boronic acid, amino, hydroxyl, haloalkyl or carboxylaye moieties. A particularly preferred substituent is an iodo substituent. The substituent or substituents having reactive functionality is/are located at the conjugated terminus or termini of the conjugated aromatic or heteroaromatic moieties. By this it is meant that the substituent(s) is/are located at the periphery of the conjugated aromatic array so that upon reaction with a suitable substrate that is itself conjugated, conjugation in the resulting product may be maintained.

Conjugated aromatic moieties useful in this embodiment of the invention include, but are not limited to, the following examples:—phenyl, naphthyl, anthracenyl, azulenyl, phenanthrenyl, tetracenyl, fluorenyl, pyrenyl, perylenyl, tetracynyl, chrysenyl, coronenyl, picenyl, pyranthrenyl, dibenzosilyl, dibenzophosphyl, carbazyl, dithienylcyclopentyl, dithienylsilyl, dithienylcarbazyl or dithienylphosphyl. A particularly preferred conjugated aromatic moiety is fluorenyl.

Advantageously the conjugated compounds of the present invention have been found to provide convenient solution processible entities. That is, they display good solubility in organic solvents. Such solubility is sufficient so to facilitate film forming processes. Surprisingly, substitution of the polyaromatic core with conjugated aromatic substituents in which the solubilising alkyl chains are attached at lateral positions in the aromatic group rather than at their terminus or termini confers good organic solvent solubility on the compound. In a particularly preferred embodiment substitution of a hexabenzocoronene (HBC) core with from two to six fluorenyl substituents (carrying 9,9-dioctyl substitution) confers good solution processibility on the HBC system and enables self organization. This is evident in the UV/VIS spectrum of the resulting film. Other structural studies (X-ray, optical microscopy, atomic force microscopy) may be used to further elucidate the self-assembled structures.

In a second aspect of the invention there is provided a compound or dendrimer formed by the reaction between the functionality on the conjugated terminus of the conjugated aromatic or heteroaromatic moiety according to the first aspect of the invention and a chain extender. Preferably, the chain extender is conjugated. More preferably, the chain extender has electron donor or acceptor characteristics. In a particularly preferred embodiment of the second aspect of the present invention the chain extender comprises triarylamine or thiophene groups.

Advantageously, in a particularly preferred embodiment of the second aspect of the present invention the aryl-functionalized HBC molecules described herein, by virtue of the unsubstituted terminus or termini, can be further chain-extended with conjugated substituents such and triaryl amines, aryl and heteroaryl groups using Suzuki, Stille, Buchwald-Hartwig, Sonogashira, Ulmann and Heck cross coupling. In principle any chain extension reaction may be applied to the conjugated terminus or termini of these molecules. A feature of the present invention is that a surprising range of substituents may be incorporated including fused and heteroatom arenes. Specifically, long chain alkyl or amphiphilic substituents are not required at the conjugated terminus. A feature of the present invention is the versatility of substitution available at the conjugated terminus. This allows the HOMO energy level to be selected and controlled. A preferred range for fullerene electron acceptor materials is −4.8 to −5.7 eV [Scharber, M. C.; Muehlbacher, D.; Koppe, M.; Denk, P.; Waldauf, C.; Heeger, A. J.; Brabec, C. J. Adv. Mater. 2006, 18, 789-794].

Any aryl-functionalised HBC compound with solubilising substituents and easily-functionalised termini has the potential to be used in organic PV devices. In a further embodiment the polycyclic aromatic or heteroaromatic cores may be extended to give larger graphitic materials. These large graphitic materials may remain solution processible and easily-functionalisable through the use of aryl or heteroaryl moieties with solubilising substituents and easily-functionalised termini. Solution processible graphitic materials have the potential to be used as transparent electrodes in organic electronic devices.

In a third aspect of the invention there is provided a hetero-junction device comprising as one active component one or more compounds or dendrimers according to any one of the embodiments of the first and second aspects of the present invention. In a particularly preferred embodiment of this aspect of the invention the device may further comprise one or more electron acceptors. Preferably, the electron acceptor is a soluble fullerene. More preferably, the electron acceptor is a C60 or C70 fullerene.

The heterojunction devices according to this aspect of the present invention may find advantageous use in a variety of electronic devices such as in light emitting diodes, transistors, photodetectors, and photovoltaic cells, for example, solar cells.

In a fourth aspect of the invention there is provided a use of a device according to the third aspect of the invention in the generation of solar power. Solar cells may be fabricated on a large scale and high solar energy efficiencies may be obtained.

Throughout this specification, use of the terms “comprises” or “comprising” or grammatical variations thereon shall be taken to specify the presence of stated features, integers, steps or components but does not preclude the presence or addition of one or more other features, integers, steps, components or groups thereof not specifically mentioned.

### BRIEF DESCRIPTION OF THE FIGURES

FIG. 1 illustrates the structures of fluorenyl-HBC cores 1, 2 and 3.

FIG. 2 illustrates: a) UV-Vis absorption spectra of FHBC derivatives 8, 12, 14 and 16 (10⁻⁵ M in CH₂Cl₂) and the UV-Vis absorption spectrum of a solid film of 16; b) UV-Vis absorption spectra of FHBC-OT hybrid 16 and thiophene dendron 9T and dendrimer 18T in CH₂Cl₂ solution (10⁻⁵ M); c) normalised UV-Vis spectra of compound 16 in CH₂Cl₂ solution at various concentrations.

FIG. 3 illustrates energy level diagrams of FHBC core 8 and FHBC-OT hybrids 12, 14 and 16, thiophene dendrimers 9T and 18T and PC₆₁BM. The data were derived from CV and UV-Vis absorption data. Note, PC₇₁BM has a similar LUMO energy level to PC₁₆BM.

FIG. 4 illustrates the concentration dependent 1H NMR spectra of compounds 8 and 14 (CDCl₃ at 20° C.). Assignment of the spectra was primarily based on the multiplicity of the peaks and by comparison with spectra of known materials.

FIG. 5 illustrates the variation in ¹H NMR chemical shift of H1 as a function of concentration for compounds 8, 12, 14 and 16. The equation is derived from the isodesmic model for stacking with equal association constants.

FIG. 6 illustrates fiber 2D-WAXS patterns of compounds a) 8 and illustration of the discotic packing, b) 14 and top view of the helical stack, c) 16 and its disordered layer organisation. The patterns were recorded at 30° C.

FIG. 7 illustrates the morphology of blend films on silicon substrate spin coated from chlorobenzene as imaged by tapping mode AFM: a) compound 8/PC₆₁BM (1:2 weight ratio); b) compound 12/PC₆₁BM (1:2 weight ratio); c) compound 14/PC₆₁BM (1:2 weight ratio) and d) compound 16/PC₆₁BM (1:2 weight ratio). The images (1×1 m) display the surface topography (height in nm).

FIG. 8 illustrates the structures of thiophene dendritic compounds used as donor materials in BHJ solar cells for comparison with FHBC-OT hybrids.

FIG. 9 illustrates a) J-V curves and b) EQE spectra of various active layer blends based devices.

FIG. 10 illustrates EQE spectra of bulk heterojunction PV cells with HBC-triarylamine dendrimer 4 and two fullerene derivatives.

### DETAILED DESCRIPTION OF THE INVENTION

It will now be convenient to describe the invention with reference to particular embodiments and examples. These embodiments and examples are illustrative only and should not be construed as limiting upon the scope of the invention. It will be understood that variations upon the described invention as would be apparent to the skilled addressee are within the scope of the invention. Similarly, the present invention is capable of finding application in areas that are not explicitly recited in this document and the fact that some applications are not specifically described should not be considered as a limitation on the overall applicability of the invention.

### HBC-Triarylamine Dendrimers

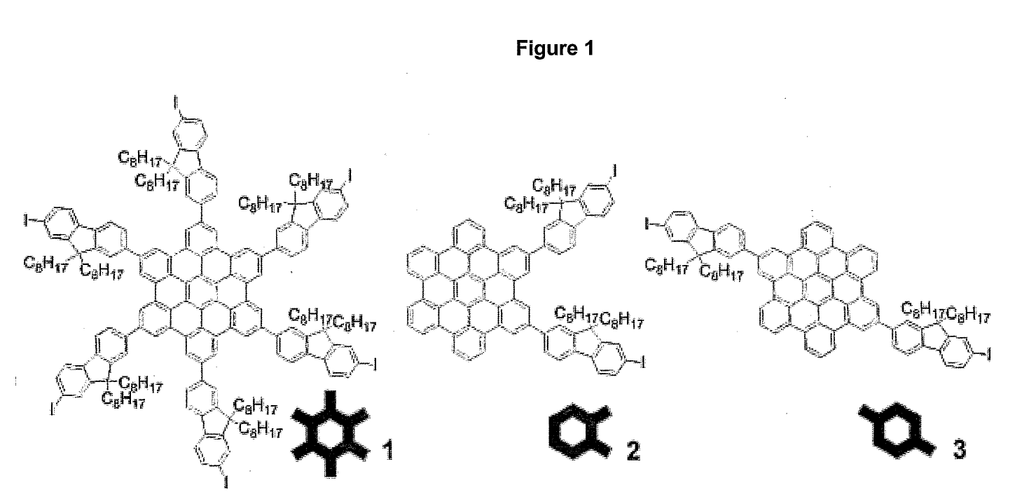

Three HBC cores have been synthesised (FIG. 1). The six-fold symmetric HBC core 1 was obtained through the Suzuki-Miyura coupling of the key asymmetric 9,9-dioctylfluorene synthon with hexa-bromophenylbenzene followed by iodination and oxidative cyclization with iron trichloride (see experimental procedures for details). HBC core 1 was highly soluble in most organic solvents and may be isolated in gram quantities in high yield. The two-fold and four-fold symmetric HBC cores 2 and 3 were also obtained in the gram scale in high yield through a series of Suzuki-Miyura coupling, aldol condensation and Diels-Alder reactions (see experimental procedures for details). Cooling of warm dichloromethane solutions of HBC cores 2 and 3 gave yellow crystalline solids which were collected by filtration. The 9,9-dioctylflorene moieties provides the solubilising property. This property makes these materials solution processible with good film forming properties.

Utilising the HBC cores illustrated in FIG. 1, electron and hole transport materials as well as dyes may be attached through the iodo-aryl functionality using a range of coupling reactions. A triarylamine oligomer 7 was coupled to the fluorenyl-HBC cores using Buchwald-Hartwig coupling. Buchwald-Hartwig coupling of the triarylamine oligomer with the HBC cores gave the three dendritic products 4, 5 and 6 in high yield (Scheme 1, see experimental procedure for details).

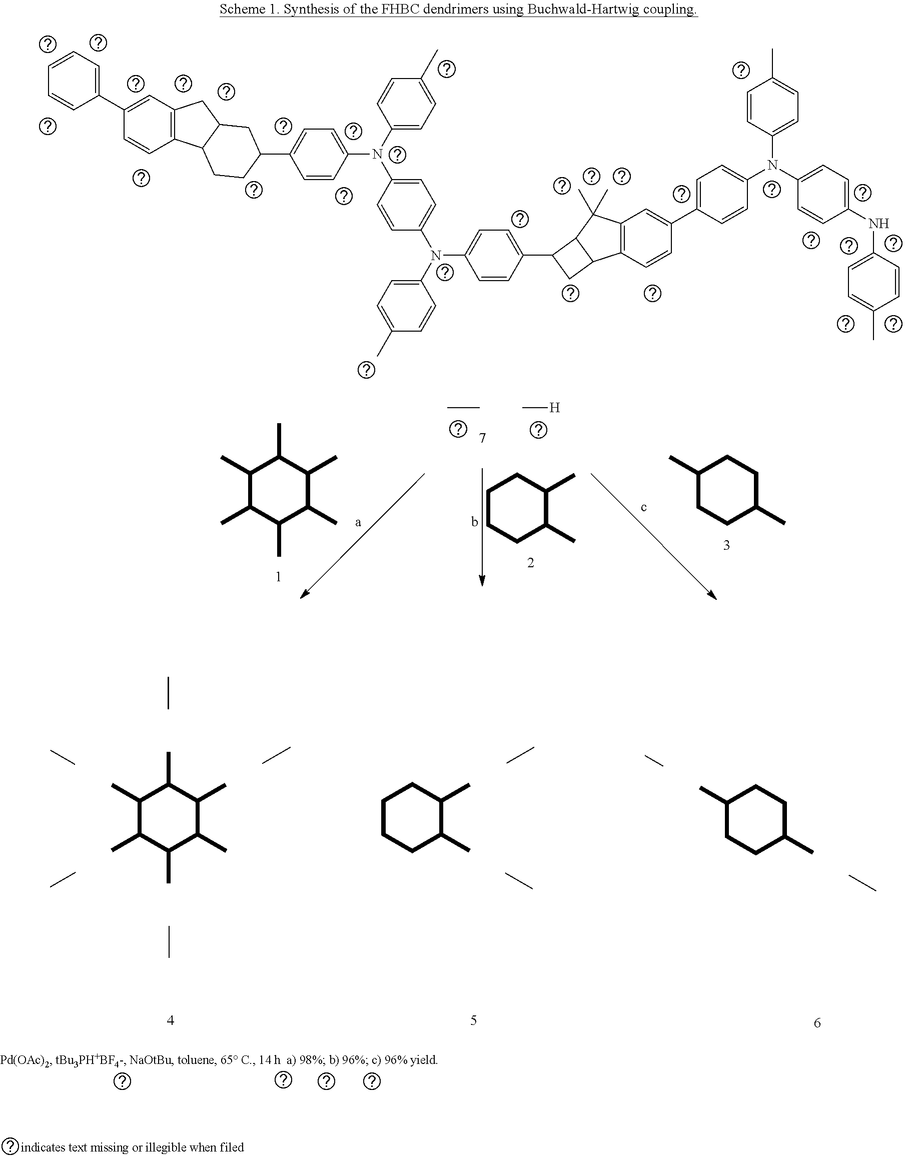

The compatibility of the HBC cores and triarylamine hole transport material was examined by fluorescence quenching studies. Thin films of HBC cores and triarylamine hole transport material and their 1:1 blends as well as the corresponding dendrimers were spincoated on glass slides (20 mg/mL toluene solution at 2000 rpm). HBC core 1 has an absorption maximum at 390 nm while cores 2 and 3 have absorption maxima at 368 and 366 nm respectively. The dendrimers obtained from the HBC cores all have similar absorption spectra with maxima at 375 nm. The fluorescence spectra of the films clearly showed the quenching of the triarylamine fluorescence in the blends and for the conjugated dendrimers. HBC core 1 quenched the fluorescence of the triarylamine completely in the blend while the fluorescence of the triarylamine was partially quenched for HBC cores 2 and 3. No fluorescence attributed to the triarylamine was observed in all three dendrimers but a weak exciplex emission at ˜540 nm was identified. This is most prominent in dendrimer 6.

The HOMO energy levels of the HBC cores 1 and 2 and dendrimers 4 and 5 were measured using electrochemical techniques. Cyclic voltammograms of these compounds were recorded in toluene solution with 0.1 M TBA BF₄ as electrolyte. Both onsets of oxidation for HBC cores 1 and 2 are at 1.0 V vs. ferrocene/ferrocenium while the oxidation onsets for dendrimers 4 and 5 are at −0.1 V. This means the HOMO levels of the HBC cores and the dendrimers are −5.8 eV and −4.7 eV respectively. The optical band gaps of all three dendrimers obtained from their thin film UV-vis spectra are approximately 2.6 eV. These energy levels confirm that the HBC dendrimers are an appropriate match with an electron acceptor, such as [6,6]-phenyl-C₆₁-butyric acid methyl ester (C₆₀ PCBM), for use in organic solar cells. HOMO energy levels can be readily measured in films using photoelectron spectroscopy in air (PESA).

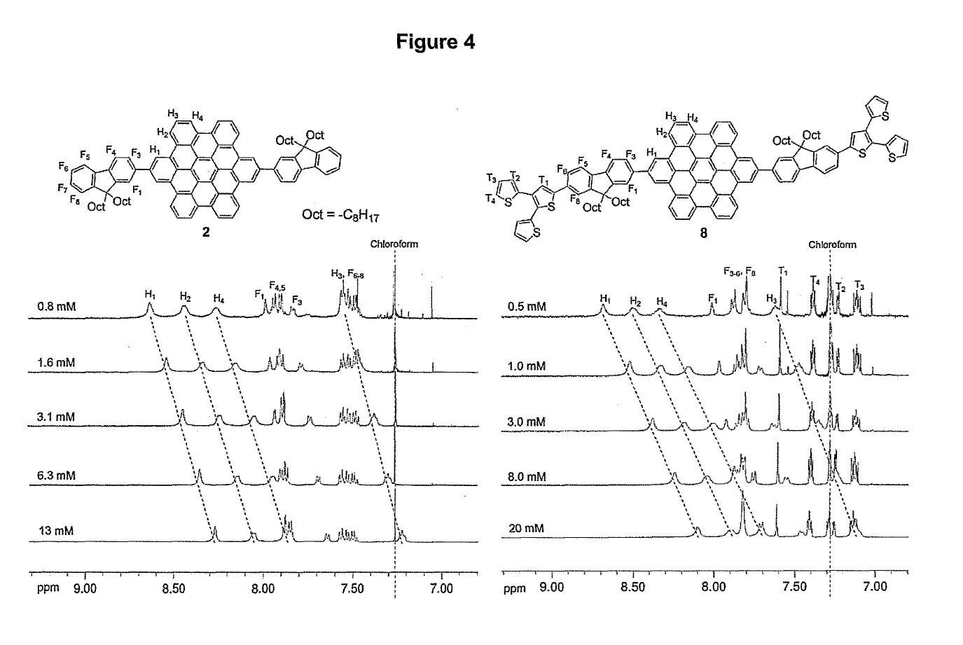

In preliminary device studies, bulk heterojunction solar cells with a device structure of ITO/PEDOT:PSS (30 nm)/active layer (40-60 nm)/Ca (20 nm)/Al (100 nm) were fabricated. The devices were tested with an Oriel solar simulator fitted with a 1000 W Xe lamp filtered to give an output of 100 mW/cm² at AM 1.5. The active layer of the device consists of a blend of one of the dendrimers and C₆₀ PCBM in ratios of 1:2 or 1:4. The performance of the devices with the three dendrimers are similar reaching V_{oc}=0.64 V, J_{sc}=0.68 mA/cm², fill factor=0.30 and power conversion efficiency=0.13%. No annealing was carried out on any of the devices. Devices consisting of the dendrimers and the C₇₀ analogue of C₆₀ PCBM were also fabricated. It has been shown that C₇₀ can provide better device performance because of its superior optical absorption profile [Wienk, M. M.; Kroon, J. M.; Verhees, W. J.; Knol, J.; Hummelen, J. C.; van Hal, P. A.; Janssen, R. A. J. Angew. Chem. Int. Ed. 2003, 42, 3371]. In a dendrimer-fullerene blend ratio of 1:2, devices with V_{oc}=0.66 V, J_{sc}=1.0 mA/cm², fill factor=0.34 and power conversion efficiency=0.22% were measured. A comparison of the IPCE spectra of the C₆₀ and C₇₀ devices clearly shows the contribution of C₇₀ to the photocurrent (see experimental section, FIG. 4). The performance of these solar cell devices are either better or comparable to literature values for devices containing HBCs [Schmidt-Mende, L.; Fechtenkotter, A.; Müllen, K.; Moons, E.; Friend, R. H.; MacKenzie, J. D. Science 2001, 293, 1119-1122; Schmidt-Mende, L.; Watson, M.; Müllen, K.; Friend, R. H. Mol. Cryst. Liq. Cryst. 2003, 396, 73-90; Hassheider, T.; Benning, S. A.; Lauhof, M. W.; Kitzerow, H. S.; Bock, H.; Watson, M. D.; Muellen, K. Mol. Cryst. Liq. Cryst. 2004, 413, 2597-2608; Jung, J.; Rybak, A.; Slazak, A.; Bialecki, S.; Miskiewicz, P.; Glowacki, I.; Ulanski, J.; Rosselli, S.; Yasuda, A.; Nelles, G.; Tomovic, Z.; Watson, M. D.; Muellen, K. Synth. Met. 2005, 155, 150-156; Schmidtke, J. P.; Friend, R. H.; Kastler, M.; Müllen, K. J. Chem. Phys. 2006, 124, 174704/1-174704/6; Li, J.; Kastler, M.; Pisula, W.; Robertson, J. W. F.; Wasserfallen, D.; Grimsdale, A. C.; Wu, J.; Müllen, K. Adv. Funct. Mater. 2007, 17, 2528-2533].

Solution processible electron acceptor materials, other than fullerenes, could also be used as is well understood in the organic PV field.

### HBC-Thiophene Dendrimers

Thiophene-based dendrons were also attached to the fluorenyl-HBC cores. Thiophene-based dendrons have been shown to function well in solution based organic PV devices with fullerenes [Ma, C.-Q.; Mena-Osteritz, E.; Debaerdemaeker, T.; Wienk, M. M.; Janssen, R. A.; Baeuerle, P. Angew. Chem. Int. Ed. 2007, 46, 1679-1683; Ma, C.-Q.; Fonrodona, M.; Schikora, M. C.; Wienk, M. M.; Janssen, R. A.; Baeuerle, P. Adv. Funct. Mater. 2008, 18, 3323-3331]. Surprisingly, these dendrons, when combined with the new aryl-extended fluorenes, demonstrate improved optical properties and device performance compared with the HBCs or dendrons alone.

The synthesis of the FHBC core 3 is given in the Examples while the thiophene dendrons 10 and 11 have been reported previously [Ma, C.-Q.; Mena-Osteritz, E.; Debaerdemaeker, T.; Wienk, M. M.; Janssen, R. A.; Baeuerle, P. Angew. Chem. Int. Ed. 2007, 46, 1679-1683]. The iodo substituents on the fluorene rings of FHBC 3 were removed using transmetallation with butyl lithium and protonation of the organolithium to give FHBC core 8 (Scheme 2). Suzuki-Miyaura coupling of the FHBC core 3 with the thiophene pinacol boronate esters 9, 10 and 11, gave, in excellent yields, the FHBC oligothiophene (FHBC-OT) hybrids 12, 13 and 15, respectively after purification by size exclusion chromatography (Scheme 2). The TMS groups of compounds 13 and 15 were removed by treatment with tetrabutylammonium fluoride which produced the desired FHBC-OT hybrids 14 and 16 in near quantitative yield (see the Examples section for full details of characterization of all new compounds). All compounds are highly soluble in organic solvents and have good film forming properties, which is desirable in the preparation of devices by solution deposition techniques.

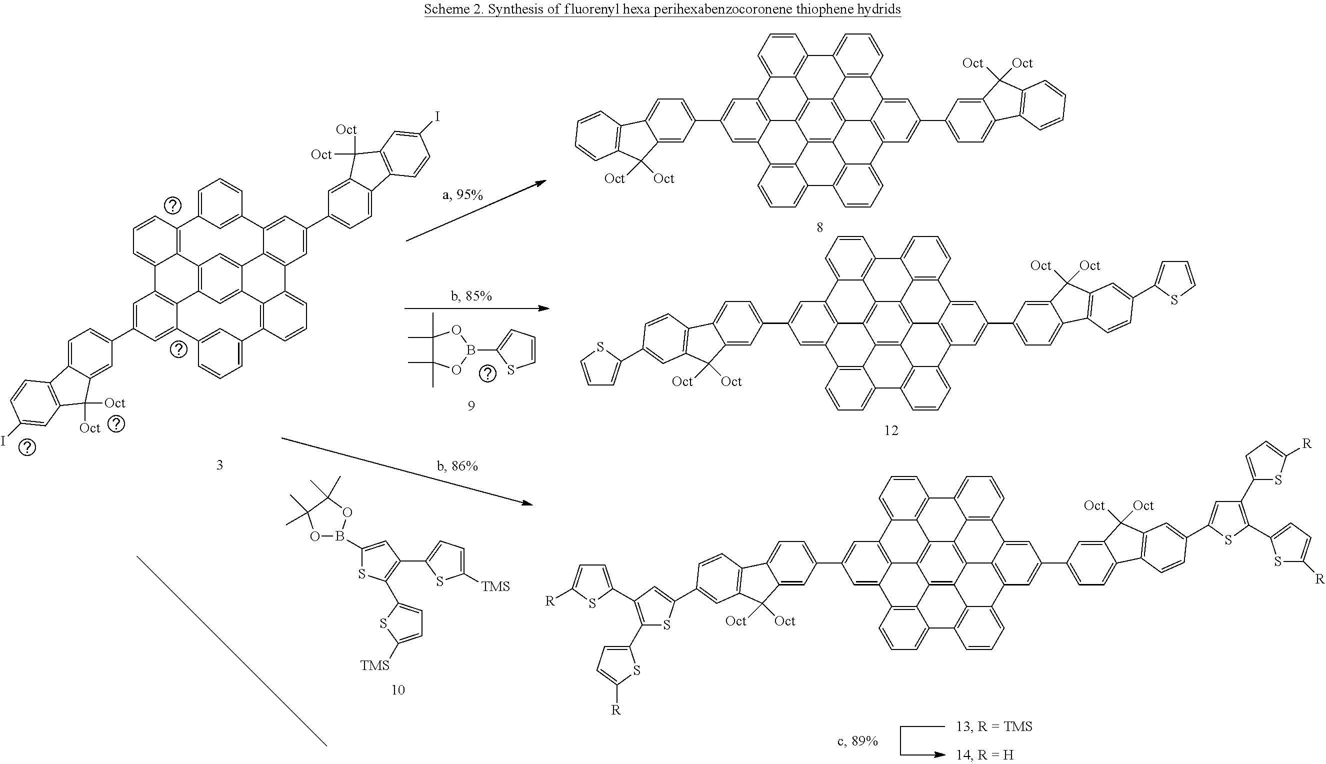

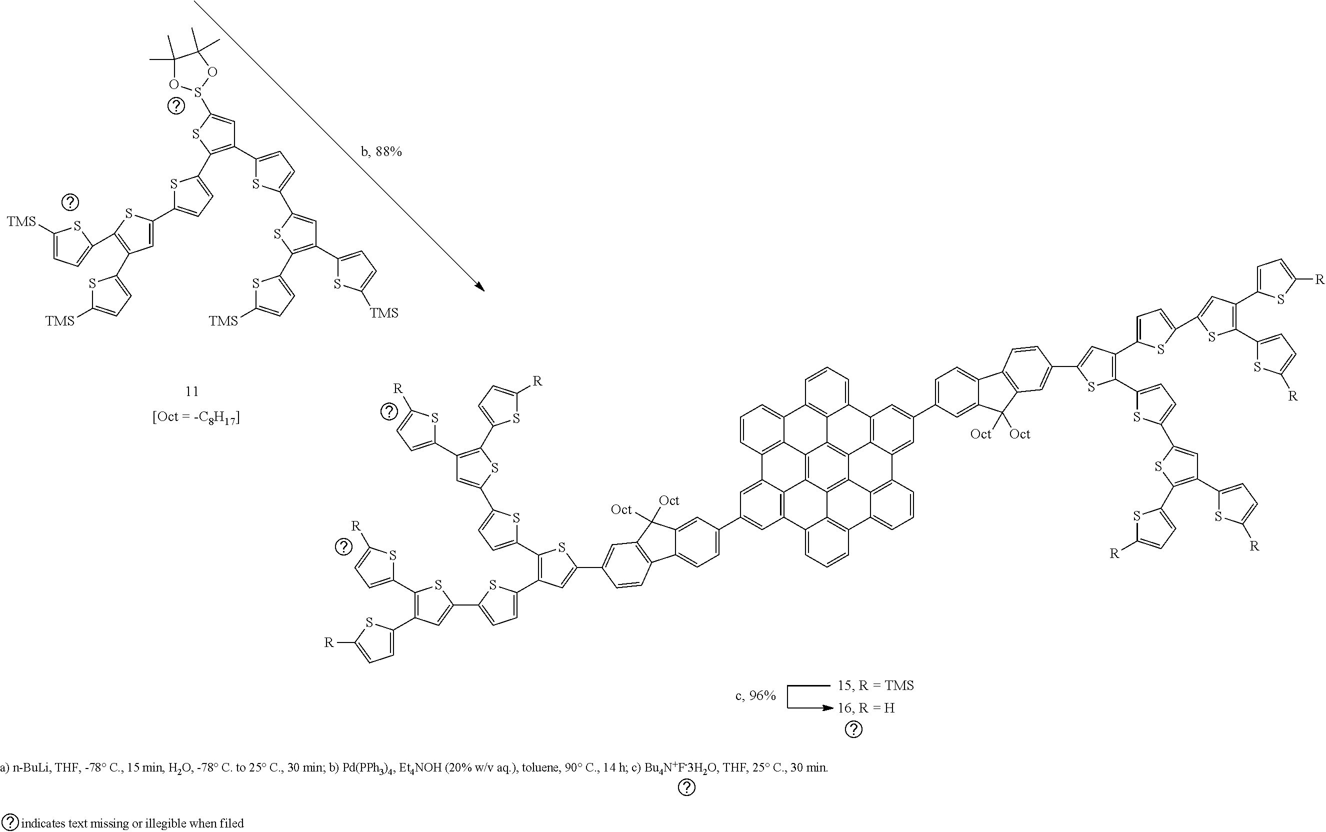

### Optoelectronic Properties

The optoelectronic properties of organic materials are important parameters that determine the applicability of a material in organic electronic devices. In bulk heterojunction solar cells, the UV-Vis absorption profile of the material is very important, as it relates to the quantity of photons the device can potentially capture. Equally important are the relative energy levels of the electron donor and acceptor materials. The energy gap between the highest occupied molecular orbital (HOMO) of the donor and the lowest unoccupied molecular orbital (LUMO) of the acceptor defines the potential output (open circuit voltage) of the device [Dennler, G.; Scharber, M. C.; Brabec, C. J. Adv. Mater. 2009, 21, 1323-1338]. In this study, the HOMO and LUMO energy levels of the materials were measured from a combination of UV-Vis spectroscopic and electrochemical techniques.

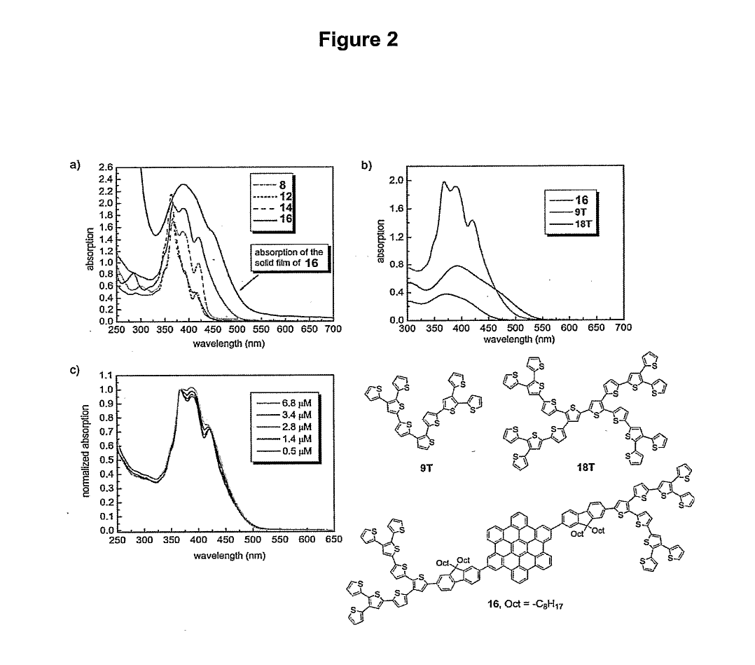

The UV-Vis spectra of FHBC core 8 and FHBC-OT hybrids 12, 14 and 16 in dichloromethane solution (10⁻⁵ M) are shown in FIG. 2a. The absorption profiles of FHBC core 8 and hybrid 12 are very similar with absorption maxima at 364 and 367 nm, respectively. The UV-Vis spectrum of 14 shows an increase in absorbance between 350 and 450 nm compared with 12. However, no red-shift was observed either for the maximum absorption wavelength or the onset absorption wavelength, indicating a lack of π-conjugation between the thiophene units and the FHBC core. Increasing the peripheral thiophene dendron size from six thiophene units in compound 14 to eighteen thiophene units in compound 16 resulted in an increased absorbance by the FHBC-OT system. The UV-Vis absorption profile of 16 is red-shifted compared with 12 and 14, with absorption onset at 500 nm. From the UV-Vis data in solution, a HOMO-LUMO gap of 2.51 eV was obtained from for 16, which agrees well with the energy gap of the second generation thiophene dendron 9T at 2.67 eV (FIG. 2b) [Ma, C.-Q.; Mena-Osteritz, E.; Debaerdemaeker, T.; Wienk, M. M.; Janssen, R. A.; Baeuerle, P. Angew. Chem. Int. Ed. 2007, 46, 1679-1683]. The fact that the second generation dendrimer 18T has a more red-shifted absorption compared with that of the FHBC-OT hybrid 16 again indicates a lack of conjugation through the entire structure of compound 16 (FIG. 2b). The break in conjugation is probably due to the relative conformation of the 9,9-dioctylfluorene units in relation to the hexa-peri-hexabenzocoronene core in compound 16. Despite this observation, compound 16 has significantly higher molar absorptivity than either 9T or 18T which may prove advantageous in solar cell devices (Table 1). The UV-Vis absorption profile of FHBC-OT hybrid 16 was recorded at a range of concentrations (FIG. 2c). The relative intensities of the absorption bands change with concentration, suggesting a degree of molecular aggregation in solution. This concentration dependence of UV-Vis spectra was also observed for compound 14. UV-Vis absorption of the thin films of all FHBC derivatives 8, 12, 14 and 16 show a shift in absorption to longer wavelengths compared with their corresponding solution spectra. For example, the absorption onset of FHBC-OT hybrid 16 as a thin film is at 550 nm compared with an onset at 500 nm in solution (FIG. 2a). This red-shift in absorption in solid state is indicative of aggregation in the solid state. The aggregation behaviour of these FHBC derivatives is discussed in greater detail in the following section using NMR spectroscopy in solution and wide angle X-ray scattering (WAXS) in solid state. Apart from increasing the UV-Vis absorption profile, the aggregation of these compounds have important effects on their solid state morphology. Morphology control in donor-acceptor blend films is crucial to the charge separation and charge transport processes that occur directly after photo-excitation in a bulk heterojunction solar cell device.

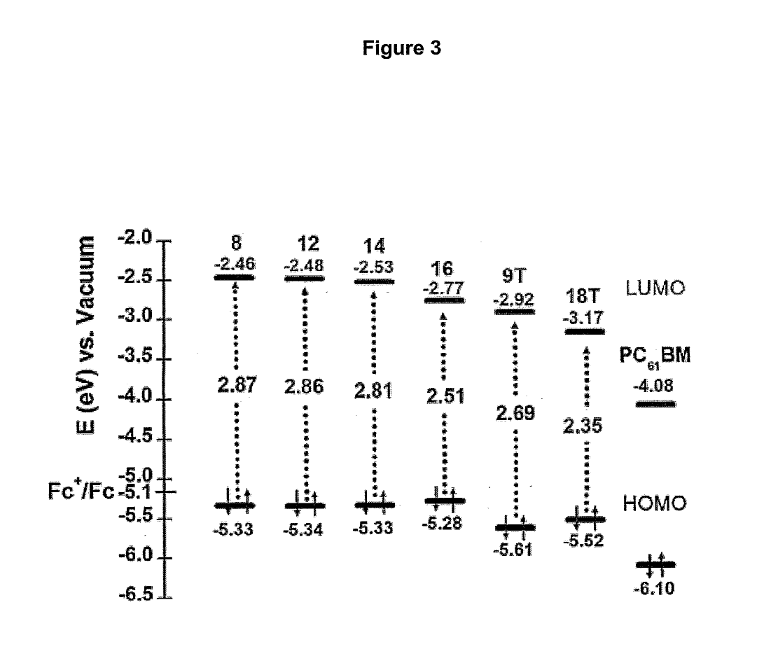

Electrochemical studies for FHBC core 8 and FHBC-OT hybrids 12, 14 and were performed in dichloromethane solution. A summary of the electrochemical data can be found in Table 1. Energy level diagrams of compounds 8, 12, 14 and 16 derived from electrochemical and UV-Vis absorption data are shown in FIG. 3. The energy level information suggests all four FHBC derivatives are suitable candidates as electron donor materials in a bulk heterojunction solar cell with [6,6]-phenyl-C61-butyric acid methyl ester (PC₆₁BM) as the electron acceptor [Scharber, M. C.; Mühlbacher, D.; Koppe, M.; Denk, P.; Waldauf, C.; Heeger, A. J.; Brabec, C. J. Adv. Mater. 2006, 18, 789-794]. Energy or charge transfer between a donor and an acceptor material can be observed by fluorescence quenching studies. The quenching of the fluorescence of the FHBC derivatives by PC₆₁BM is another indication of compatibility of the materials for use in BHJ solar cell devices. In thin films, the fluorescence of the FHBC derivatives was completely quenched when blended in a 1:2 weight ratio of FHBC to PC₆₁BM.

TABLE 1
Optical and redox data of the FHBC core 8 and HBC-OT hybrid compounds.

| | λ_{abs}^{max} | ε × 10⁵ | E_{g}^{opt} | Eₒₓ¹ | Eₒₓ^{onset} | HOMO | LUMO |
|---|---|---|---|---|---|---|---|
| Compounds | (nm)^{[a]} | (cm · L · mol⁻¹)^{[a]} | [eV]^{[a,b]} | [V]^{[c,d]} | [V]^{[c]} | [eV]^{[e]} | [eV]^{[f]} |
| 8 | 364 | 2.15 | 2.87 | 0.24 | 0.23 | −5.33 (−5.28) | −2.46 |
| 12 | 367 | 1.75 | 2.86 | 0.32 | 0.24 | −5.34 (−5.37) | −2.48 |
| 13 | 367 | 1.71 | 2.79 | 0.31 | 0.22 | −5.32 (−5.27) | −2.53 |
| 14 | 368 | 1.69 | 2.81 | 0.31 | 0.23 | −5.33 (−5.38) | −2.52 |
| 15 | 367 | 2.17 | 2.47 | 0.24 | 0.22 | −5.32 (−5.19) | −2.85 |
| 16 | 369 | 1.98 | 2.51 | 0.24 | 0.18 | −5.28 (−5.40) | −2.77 |
| 9T | 373 | 0.38 | 2.69 | 0.57 | 0.51 | −5.61 | −2.92 |
| 18T | 392 | 0.78 | 2.35 | 0.46^{[g]} | 0.42^{[g]} | −5.52^{[g]} | −3.17 |

^{[a]}in CH₂Cl₂, 1 × 10⁻⁵ M, 295K;
^{[b]}determined from the onset of absorption;
^{[c]}in CH₂Cl₂, 1 × 10⁻³ M, Bu₄NPF₆ (0.1M), 295K, scan rate = 100 mV · s⁻¹, versus Fc/Fc⁺;
^{[d]}determined by differential pulse voltammetry;
^{[e]}determined from E_{HOMO} = (Eₒₓ^{onset} + 5.10) (eV), [Scharber, M. C.: Mühlbacher, D.; Koppe, M.; Denk, P.; Waldauf, C,; Heeger, A. J.; Brabec, C. J. Adv. Mater. 2006, 18, 789-794] data in brackets measured by photoelectron spectroscopy in air; [Kane, E. O. Physical Review 1962, 127, 131-141; Kirihata, H.; Uda, M. Rev. Sci. Instrum. 1981, 52, 68-70]
^{[f]}calculated from LUMO = HOMO + E_{g}^{opt};
^{[g]}in DMF, 1 × 10⁻⁴ M, Bu₄NPF₆ (0.1M), 295K, scan rate = 100 mV · s⁻¹, versus Fc/Fc⁺.

### Self-Association Properties and Solid State Morphology

As mentioned in the discussion of the UV-Vis absorption experiments above, aggregation behaviour was observed in solution and in the solid state. While many molecular systems will aggregate in solution given the appropriate solvation conditions, the ordered association of molecules requires correct molecular design. Planar aromatic systems, like hexa-peri-hexabenzocoronene (HBC), chiefly rely on π-π stacking as the force for association. In fact, the poor solubility of unsubstituted HBC is a consequence of this strong π-π stacking association. The fluorenyl HBC derivatives in this study rely on the 9,9-dioctylfluorene units to impart solubility. The steric bulk of the 9,9-dioctylfluorene groups limit extended aggregation compared with unsubstituted HBC. However, the 2,11-disubstitution arrangement on the HBC molecule with the fluorenyl groups as in compounds 8, 12, 14 and 16 still allows π-π stacking of the HBC core. This phenomenon can be directly observed by NMR spectroscopic studies in solution.

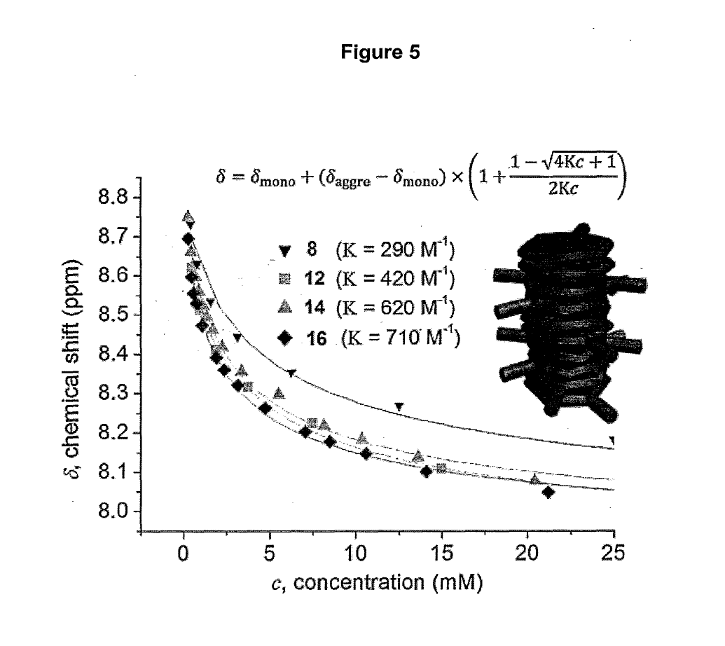

¹H NMR spectra of the aromatic region for compounds 8 and 14 at various concentrations are shown in FIG. 4. Peak assignments were made primarily on the basis of the multiplicity of the peaks and by comparison with spectra of known material. The ¹H NMR spectra of the FHBC core 8 and FHBC-OT hybrids 12-16 were found to be concentration dependent. It is clear that the protons assigned to the HBC core (H₁₋₄) shift upfield with increasing concentration (FIG. 4). The protons on the fluorene moiety which are closest to the core (F₁ and F₃) also shift upfield with increasing concentration. The upfield shift of these protons is likely due to a shielding effect caused by staggered π-π stacking between FHBC-OT molecules (FIGS. 4 and 5). The fact that the protons on the thiophene moiety do not show changes in chemical shift as a function of concentration supports this staggered π-π stacking model. An isodesmic model of indefinite stacking can be fitted to the changes in chemical shift with concentration [Martin, R. B. Chem. Rev. 1996, 96, 3043-3064]. Association constants (K) were obtained by fitting the data to the equation for isodesmic model for stacking with equal association constants [Martin, R. B. Chem. Rev. 1996, 96, 3043-3064]. The chemical shift of the H₁ proton of the unassociated monomer (δₘₒₙₒ) was arbitrarily set at 9 ppm while that of the aggregate (δ_{aggre}) was arbitrarily set at 8 ppm. Plots of concentration versus chemical shift for compounds 8, 12, 14 and 16 follow a similar trend and the data fit well (R²>0.99) with the proposed indefinite stacking model (FIG. 5). It is interesting to note that the increase in thiophene dendron size does not appear to have an adverse effect on the proposed π-π stacking association of the HBC core. In fact, there appears to be an increase in association with increasing dendron size. However, the significance of this observation is uncertain as the calculated deviation on the association constant is close to ±20%. In any case, the results in these NMR studies support the observations made in the UV-Vis spectroscopic studies confirming a self-association behaviour in solution.

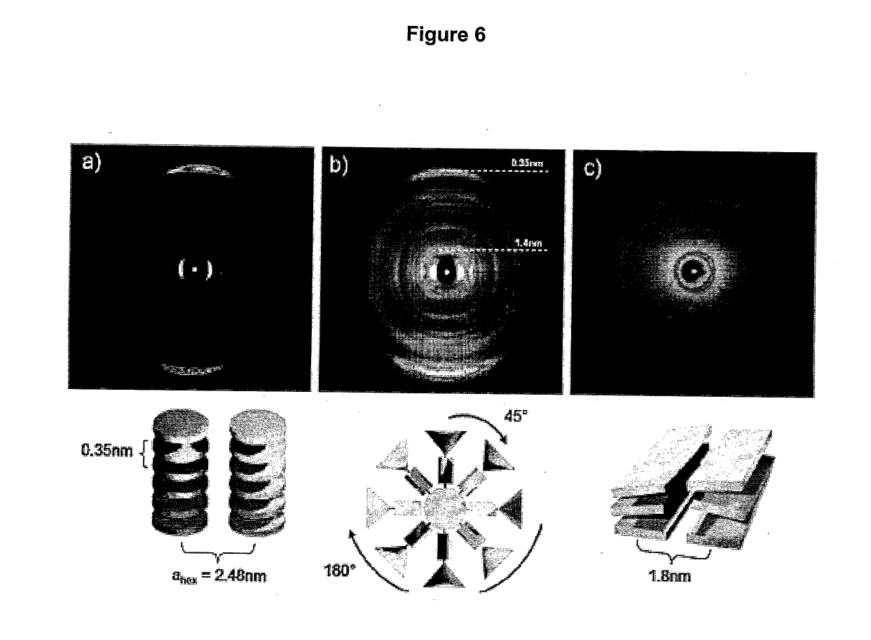

While the above discussed NMR results indicate self-association in solution, X-ray scattering experiments provide information about the organization and phase formation in the solid state. Two-dimensional wide-angle X-ray scattering (2D-WAXS) experiments were performed on thin filaments of compounds 8, 14 and 16. Filaments of 0.7 mm diameter were prepared by filament extrusion and mounted vertical towards the 2D detector. FIG. 6a shows a 2D pattern for 8 which is characteristic for a discotic columnar liquid crystalline phase [Laschat, S.; Baro, A.; Steinke, N.; Giesselmann, F.; Hägele, C.; Scalia, G.; Judele, R.; Kapatsina, E.; Sauer, S.; Schreivogel, A.; Tosoni, M. Angew. Chem. Int. Ed. 2007, 46, 4832-4887; Sergeyev, S.; Pisula, W.; Geerts, Y. H. Chem. Soc. Rev. 2007, 36, 1902-1929]. The equatorial reflections indicate an orientation of the columnar stacks along the fiber alignment direction. A hexagonal columnar arrangement with a unit cell of aₕₑₓ=2.48 nm for 8 was determined from the relative reciprocal spacing of 1:\sqrt{3}:2 of the scattering intensities. The distinct meridional reflections in the wide-angle region are attributed to the cofacial π-stacking distance of 0.35 nm between individual molecules within the column. Thereby, the discs are packed with their molecular planes perpendicular to the columnar axis as illustrated schematically in FIG. 6a. This liquid crystalline organization remains unchanged over the whole investigated temperature range of −100° C. b 200° C., and is in agreement with the thermal analysis by differential scanning calorimetry (DSC), which did not reveal any phase transitions. Similarly, compound 14 showed no transitions in the DSC scans. The structural analysis for 14 pointed towards a rectangular columnar organization with unit cell dimensions of a=2.56 nm and b=1.91 nm. The significantly smaller unit cell in comparison to the theoretical molecular length (ca. 4.2 nm) is related to only two substituents (low density of the substitution mantel around the HBC stack) and thus intercalation of these substituents between neighbouring columns. A π-stacking distance of 0.35 nm was also determined for 14 from the wide-angle meridional scattering intensity. In strong contrast to the behaviour of FHBC 8, the appearance of additional meridional reflections for compound 14 is characteristic of a complex helical packing of the molecules within the stacks [Hoist, H. C.; Pakula, T.; Meier, H. Tetrahedron 2004, 60, 6765-6775; Pisula, W.; Kastler, M.; Wasserfallen, D.; Robertson, J. W. F.; Nolde, F.; Kohl, C.; Muellen, K. Angew. Chem. Int. Ed. 2006, 45, 819-823; Livolant, F.; Levelut, A. M.; Doucet, J.; Benoit, J. P. Nature (London, United Kingdom) 1989, 339, 724-726; Percec, V.; Imam, M. R.; Peterca, M.; Wilson, D. A.; Heiney, P. A. J. Am. Chem. Soc. 2009, 131, 1294-1304; Peterca, M.; Percec, V.; Imam, M. R.; Leowanawat, P.; Morimitsu, K.; Heiney, P. A. J. Am. Chem. Soc. 2008, 130, 14840-14852; Lehmann, M.; Jahr, M.; Donnio, B.; Graf, R.; Gemming, S.; Popov, I. Chem. Eur. J. 2008, 14, 3562-3576]. The position of the middle-angle reflection indicated in FIG. 6b is related to an additional period of 1.4 nm between every 4^{th} molecule (1.4 nm/0.35 nm=4) along the column possessing identical positional order. Thereby, the discs are substantially rotated by 45° b each other, while the aromatic HBC cores are perpendicular to the columnar axis. The additional meridional intensities at multiple scattering angles are higher order reflections. This kind of helical arrangement in a so-called plastic phase is in agreement with other discotic molecules bearing bulky substituents which induce a lateral rotation of neighboring discs [Vera, F.; Serrano, J.-L.; Sierra, T. Chem. Soc. Rev. 2009, 38, 781-796; Barberá, J.; Cavero, E.; Lehmann, M.; Serrano, J.-L.; Sierra, T.; Vázquez, J. T. J. Am. Chem. Soc. 2003, 125, 4527-4533; Percec, V.; Imam, M. R.; Peterca, M.; Wilson, D. A.; Graf, R.; Spiess, H. W.; Balagurusamy, V. S. K.; Heiney, P. A. J. Am. Chem. Soc. 2009, 131, 7662-7677; Feng, X.; Wu, J.; Ai, M.; Pisula, W.; Zhi, L.; Rabe, J. P.; Müllen, K. Angew. Chem. Int. Ed. 2007, 46, 3033-3036; Pisula, W.; Tomović, \check{Z}.; Watson, M. D.; Müllen, K.; Kussmann, J.; Ochsenfeld, C.; Metzroth, T.; Gauss, J. J. Phys. Chem. B 2007, 111, 7481-7487; Feng, X.; Pisula, W.; Müllen, K. J. Am. Chem. Soc. 2007, 129, 14116-14117; Feng, X.; Marcon, V.; Pisula, W.; Hansen Michael, R.; Kirkpatrick, J.; Grozema, F.; Andrienko, D.; Kremer, K.; Müllen, K. Nature Materials 2009, 8, 421-426; Fontes, E.; Heiney, P. A.; De Jeu, W. H. Phys. Rev. Lett. 1988, 61, 1202-1205]. Typically, such helical organization in liquid crystalline columnar stacks vanishes at high temperatures, but this complex arrangement for compound 14 remained unchanged at 160° C. indicating pronounced stability of the plastic phase within a broad temperature range. The direct comparison of the intracolumnar packing between 8 and 14 indicates that the helical stacking originates from the additional sterically demanding thiophene dendrons on compound 14. The increase of the steric hindrance by attaching even larger 9T dendrons for dendrimer 16 resulted in a more disordered structure in the bulk. The isotropic reflection corresponding to a distance of 1.8 nm is attributed to the spacing between lamellar layers which are formed by local phase separation between the rigid aromatic part and flexible side chains (FIG. 6c). The molecules within the lamellar structures of 16 are much more disordered compared to the molecules in the columnar packing of FHBC 8 and 14. These structural parameters are reflected in the BHJ solar cell performance characteristics of these materials and will be discussed in the following section.

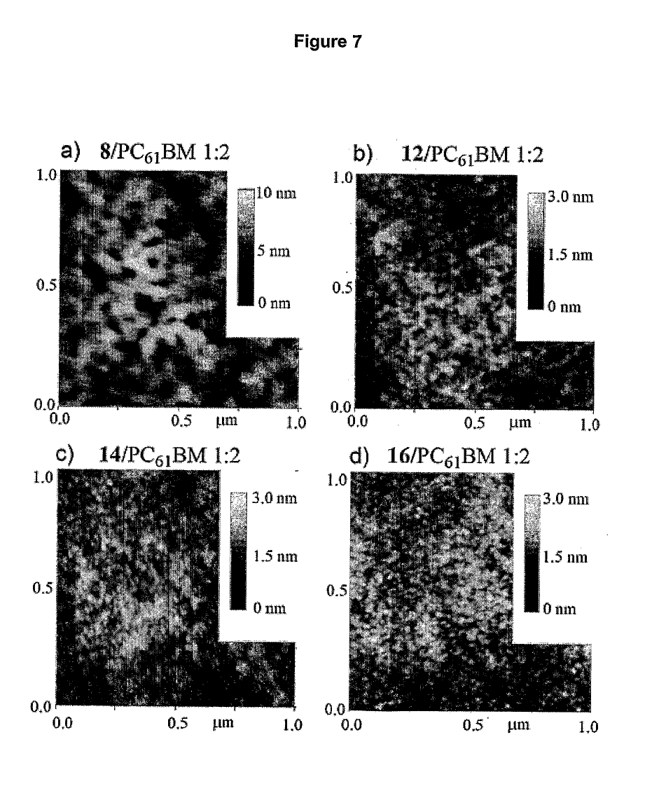

The surface morphology of thin films was examined using tapping mode atomic force microscopy (AFM). The samples were prepared by spin coated the material of interest on silicon substrate (25 mg/mL in chlorobenzene, 2000 rpm). The tapping mode AFM images of thin films of blends of compounds 8, 12, 14 and 16 with PC₆₁BM (1:2) are shown in FIG. 6. Nano-scale phase separation was observed in all four blend films. The blend of 8 and PC₆₁BM film gave the largest phase separation with domain sizes of ˜100 nm (FIG. 7). The phase domains were smaller for blend films of 12, 14 and 16 with PC₆₁BM and smoother film surfaces were observed. These differences in film morphology have consequences to device performance and will be discussed in the following section. Pristine films of compounds 8, 12, 14 and 16 were also examined using tapping mode AFM. The surface roughness of films containing compounds 8 and 14 was much higher than the roughness of the film containing compound 16. This is in agreement with the results obtained in the 2D-WAXS experiments where higher molecular order and crystallinity was observed for compounds 8 and 14 compared to compound 16 (FIG. 6).

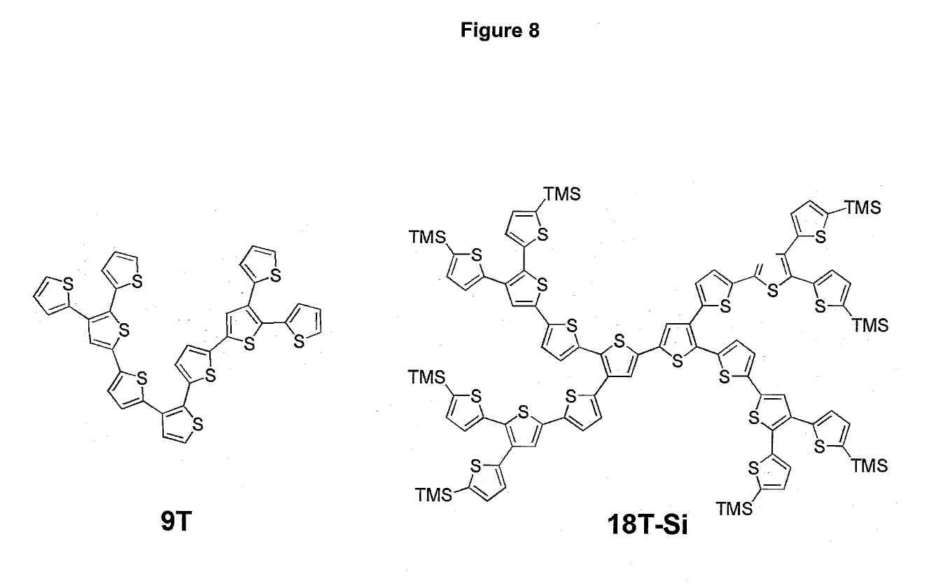

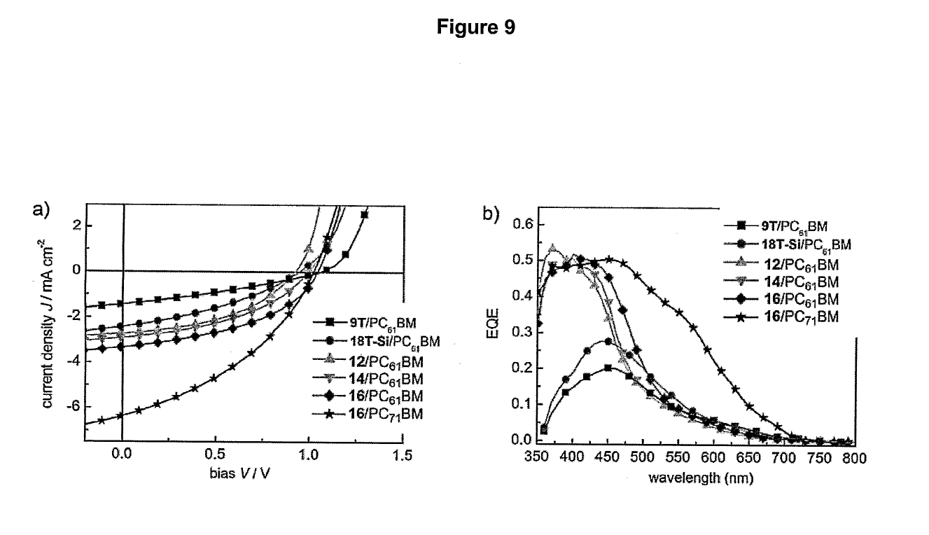

In light of the foregoing photophysical and self-organization studies, the FHBC derivatives appear ideal candidates to be employed as the electron donor material in BHJ solar cells. Accordingly, BHJ solar cells with device structure ITO|PEDOT:PSS|FHBC-OT:fullerene (1:2 w/w)|LiF/Al [ITO, indium tin oxide; PEDOT, poly(3,4-ethylenedioxythiophene); PSS, poly(styrenesulfonate)] using the FHBC-OT hybrids 12, 14 and 16 as electron donors, and fullerene derivatives as electron acceptor were fabricated and characterized. Devices with compound 8, using Ca instead of LiF at the Al cathode, were also fabricated and tested. The ratio of donor and acceptor materials was device-optimized at 1:2 and is in line with the fluorescence quenching studies. The thickness of the photoactive layers was optimized for each of the donor-acceptor blends and was typically between 60 and 70 nm. In general, all devices showed good diode-like behaviour in the dark and photovoltaic effects under simulated AM 1.5G illumination. Table 2 summarizes the device performance of the various solar cells and the following characteristic parameters are given: short-circuit currents (J_{sc}), open-circuit voltages (V_{oc}), fill factors (FF), and power-conversion efficiencies (η). For comparison, the PV performance data of the all-thiophene dendron 9T and the dendrimer 18T-Si are shown in Table 2 and has been reported previously [Ma, C.-Q.; Fonrodona, M.; Schikora, M. C.; Wienk, M. M.; Janssen, R. A. J.; Bäuerle, P. Adv. Funct. Mater. 2008, 18, 3323-3331]. The structures of these thiophene dendritic materials are shown in FIG. 8. Devices containing 18T could not be fabricated due to the low solubility of 18T in commonly used solvents. It should be noted that the device-optimized weight ratio between donors 9T and 18T-Si and PC₆₁BM was 1:4. The difference in device-optimized weight ratio between the FHBC and thiophene dendron-based devices can be related to the morphology of the device films. In the case of the thiophene dendrons 9T and 18T-Si, more PC₆₁BM is required for optimal phase separation, leading to an interpenetrating network morphology required for efficient device operation. On the other hand, there is clear phase-separation between the donor and acceptor domains in FHBC/PC₆₁BM (1:2 w/w) blends as observed in AFM experiments (FIG. 7). The current density to voltage and external quantum efficiency curves for the BHJ devices are shown in FIG. 9.

High open-circuit voltages (V_{oc}) of 0.9 to 1.0 V were observed for all compound combinations. The V_{oc} of a BHJ solar cell device depends primarily on the energy gap between donor HOMO and acceptor LUMO of the materials. Energy gaps of 1.2 to 1.3 eV, derived from FIG. 3, are in agreement with the V_{oc} values measured for the devices. These V_{oc} values are also comparable to that of pure thiophene dendrimers 9T and 18T-Si recently reported [Ma, C.-Q.; Fonrodona, M.; Schikora, M. C.; Wienk, M. M.; Janssen, R. A. J.; Bäuerle, P. Adv. Funct. Mater. 2008, 18, 3323-3331] and is considerably better than typical V_{oc} of P3HT:PC₆₁BM BHJ solar cells (0.55-0.65 V) [Ma, W.; Yang, C.; Gong, X.; Lee, K.; Heeger, A. J. Adv. Funct. Mater. 2005, 15, 1617-1622]. A clear trend was observed for the short-circuit currents (J_{sc}) of the series of devices. The value of J_{sc} increased with the broadening of the optical absorption from the zero generation dendrimer 12 to the second generation dendrimer 16. The short circuit current J_{sc} of compound 16 based device (Table 2, entry 4) is much higher than that of the corresponding thiophene dendron 9T (entry 6) and dendrimer 18T-Si (entry 7). This is likely due to the much higher absorption of 16 over 350-450 nm originating from the FHBC core (FIG. 2b). In this study, the best fill factor (FF) of 0.54 was observed for the device containing the FHBC core 8. A good FF indicates efficient as well as balanced charge transport within the active layer of the device. The ordered assembly of compound 8 in the solid state, as demonstrated by 2D-WAXS (FIG. 6a), will almost certainly facilitate charge transport. The FF for the device containing 10 (entry 4) is higher than the 9T and 18T-Si devices (entry 6 & 7). This can be rationalized by the better charge carrier transport within the active layer induced by ordered assembly of the FHBC core moiety. The value of J_{sc} was also improved significantly by the use of PC₇₁BM instead of PC₆₁BM (compare entries 4 & 5 in Table 2) [Wienk, M. M.; Kroon, J. M.; Verhees, W. J. H.; Knol, J.; Hummelen, J. C.; van Hal, P. A.; Janssen, R. A. J. Angew. Chem. Int. Ed. 2003, 42, 3371-3375]. PC₇₁BM has increased optical absorption compared to PC₆₁BM and has been shown to improve light harvesting in organic solar cells. External quantum efficiency (EQE) spectra show the photo-current response of the devices at wavelengths from 350 to 850 nm (FIG. 9b). A maximum EQE of 50% was obtained for devices with PC₆₁BM at around 400 nm. The maximum EQE of the device containing the FHBC-OT hybrid 16 and PC₇₁BM was extended to 470 nm. A power conversion efficiency of 2.5% was achieved for the device with minimal optimization in the active layer thickness, donor-acceptor ratio and morphology.

In summary, the addition of the FHBC core to the thiophene dendrimers improved the performance of the material in BHJ solar cells. The FHBC core increased the photocurrent generated from the solar cells by absorbing more strongly over 350-450 nm compared to the pure thiophene dendrimers (FIGS. 2b and 9b). In addition, the self-assembling properties of the FHBC core drives the formation of ordered morphology in solid state. The 2D-WAXS experiments showed self-assembly of the FHBC material into ordered structures (FIG. 6) while tapping mode AFM studies indicate nano-scale phase separation between the donor and acceptor domains in blend films (FIG. 7). The combination of nano-scale donor-acceptor phase separation and the formation of ordered structures within these domains are important to charge separation and transport in the active layer of the solar cells after photoexcitation.

TABLE 2
Device performance of bulk heterojunction solar cells (see text)
with active layers consisting of dendrimer/fullerene 1:2 blends.
Active layer thickness was 60-70 nm and device area was 0.167 cm².

| | | acceptor | J_{sc}^{[a]} | V_{oc} | | η^{[b]} |
|---|---|---|---|---|---|---|
| entry | donor | (weight ratio) | (mA cm⁻²) | (V) | FF | (%) |
| 1 | 8^{[c]} | PC₆₁BM (1:2) | 1.87 | 0.9 | 0.54 | 0.9 |
| 2 | 12 | PC₆₁BM (1:2) | 2.73 | 0.9 | 0.44 | 1.1 |
| 3 | 14 | PC₆₁BM (1:2) | 2.91 | 1.0 | 0.42 | 1.2 |
| 4 | 16 | PC₆₁BM (1:2) | 3.33 | 1.0 | 0.44 | 1.5 |
| 5 | 16 | PC₇₁BM (1:2) | 6.37 | 1.0 | 0.38 | 2.5 |
| 6 | 9T^{[d]} | PC₆₁BM (1:4) | 1.42 | 1.0 | 0.31 | 0.5 |
| 7 | 18T-Si^{[d]} | PC₆₁BM (1:4) | 2.39 | 0.9 | 0.35 | 0.8 |

^{[a]}Determined by convoluting the spectral response with the AM 1.5G spectrum (100 mW cm⁻²);
^{[b]}η = J_{sc} × V_{oc} × FF;
^{[c]}Ca was used instead of LiF for this device;
^{[d]}from reference [Ma, C.-Q.; Fonrodona, M.; Schikora, M. C.; Wienk, M. M.; Janssen, R. A. J.; Bäuerle, P. Adv. Funct. Mater. 2008, 18, 3323-3331].

### Summary

The design of novel materials for organic solar cell applications is currently a topic of great interest. While it is important to maximize the harvesting of sunlight by broadening the absorption profile of organic materials, it is also essential that the light energy absorbed by the material is efficiently converted into electric current. An interpenetrating network of donor and acceptor materials with domain size of 15-20 nm is thought to be ideal for charge separation and charge transport after photo-excitation in a bulk heterojunction (BHJ) solar cell device. Molecular organization within the donor and acceptor domains is also important for charge transport. In this study, fluorenyl hexa-peri-hexabenzocoronene (FHBC) was employed as the scaffold for molecular organization. FHBC derivatives with various dendritic thiophene substituents have been shown to self-associate into ordered structures in solution and in solid state. BHJ solar cell devices fabricated with these compounds as electron donor materials show good performance achieving power conversion efficiency of 2.5%. In addition, a comparison of devices based on the FHBC derivatives and pure dendritic thiophene materials showed the positive effect of self-organization on device performance.

### EXAMPLES

All reactions were performed using anhydrous solvent under an inert atmosphere unless stated otherwise. Silica gel (Merck 9385 Kieselgel 60) was used for flash chromatography. Thin layer chromatography was performed on Merck Kieselgel 60 silica gel on glass (0.25 mm thick). ¹H and ¹³C NMR spectroscopy were carried out using either a Varian Inova-400 (400 MHz) or the Varian Inova-500 (500 MHz) instruments. Mass spectra were obtained by the mass spectrometry service at CSIRO MHT at Clayton (EI) and the EPSRC mass spectrometry centre in Swansea (MALDI). IR spectra were obtained on a Perkin Elmer Spectrum One FT-IR spectrometer while UV-vis spectra were recorded using a Cary 50 UV-vis spectrometer. Photoluminescence was measured with a Varian Cary Eclipse fluorimeter. Melting points were determined on a Büchi 510 melting point apparatus. Elemental analyses were obtained commercially through CMAS, Victoria. 2-Pinacolborolane-7-trimethylsilyl-9,9-dioctylfluorene [Sandee, A. J.; Williams, C. K.; Evans, N. R.; Davies, J. E.; Boothby, C. E.; Köhler, A.; Friend, R. H.; Holmes, A. B. J. Am. Chem. Soc. 2004, 126, 7041], thiophene dendrons 10 and 11 [Ma, C.-Q.; Mena-Osteritz, E.; Debaerdemaeker, T.; Wienk, M. M.; Janssen, R. A.; Baeuerle, P. Angew. Chem. Int. Ed. 2007, 46, 1679-1683; Ma, C.-Q.; Fonrodona, M.; Schikora, M. C.; Wienk, M. M.; Janssen, R. A.; Baeuerle, P. Adv. Funct. Mater. 2008, 18, 3323-3331], compound 24 [Watanabe, S.; Kido, J. Chem. Lett. 2007, 36, 590], N-4-bromophenyl-tolylaniline and tert-butyl 4-bromophenyl(p-tolyl)carbamate [Brown, B. A.; Leeming, S. W.; Williams, R. Triarylamine compounds, compositions and devices, WO2006010555 (A1), 2006, CAN 144:203501] have been reported in the literature. All other compounds and reagents are commercially available.

### Two-Dimensional Wide-Angle X-Ray Scattering

The WAXS experiments were performed using a Rigaku 18 kW rotating copper anode as source, and a double graphite monochromator to give CuK_{α} radiation (λ=1.54 Å). The X-ray beam was collimated using pinholes, and the scattered radiation was collected using a two-dimensional Siemens detector. The samples were prepared by filament extrusion using a home-built mini-extruder. Therein, if necessary, the material is heated up to a phase at which it becomes plastically deformable and is extruded as 0.7 mm thin fiber by a constant-rate motion of the piston along the cylinder.

### Tapping Mode Atomic Force Microscopy

Tapping mode AFM (NanoScope II, Dimension, Digital Instrument Inc.) was carried out with commercially available tapping mode tips. The scanning area is between 10×10 μm² and 1×1 μm². The AFM samples were prepared by spin casting the material of interest (25 mg/mL in chlorobenzene, 2000 rpm) on silicon substrate.

### Example 1

### HBC Core 1 (See Scheme 3)

Compound 14 (1 g, 0.28 mmol) was dissolved in dry CH₂Cl₂ (250 mL) and the solution was degassed by bubbling argon through. A solution of iron(III) chloride (0.8 g, 5 mmol) in dry nitromethane (10 mL) was added to the solution with argon bubbling through the reaction. The reaction was stirred for 45 min at 25° C. and the solvent was removed under vacuum. The product was isolated as a yellow powder (0.9 g, 90% yield) after purification by column chromatography (SiO₂, Pet. spirit 40-60/CH₂Cl₂ 4:1, R_{f} 0.5). m.p. 158-160° C.

¹H NMR (500 MHz, CDCl₃, δ): 0.76 (br m, 36H, —CH₃), 0.84 (br m, 24H, —CH₂—), 1.14 (br m, 120H, —CH₂—), 2.14 (br m, 24H, —CH₂—), 7.56 (br, 6H, ArH), 7.75-8.12 (br m, 30H, ArH), 9.31-9.64 (br m, 12H, ArH). ¹³C NMR (125 MHz, CDCl₃, δ): 14.3, 22.9, 24.2, 29.5, 30.2, 30.4, 32.0, 40.7, 55.9, 93.1, 120.9, 122.0, 122.4, 127.4, 131.2, 132.5, 136.4, 140.5, 141.3, 151.7, 153.7. MS-MALDI (m/z): M⁺ 3609.4. Elemental analysis: cal. C, 71.87; H, 7.04. found C, 70.80; H, 6.78.

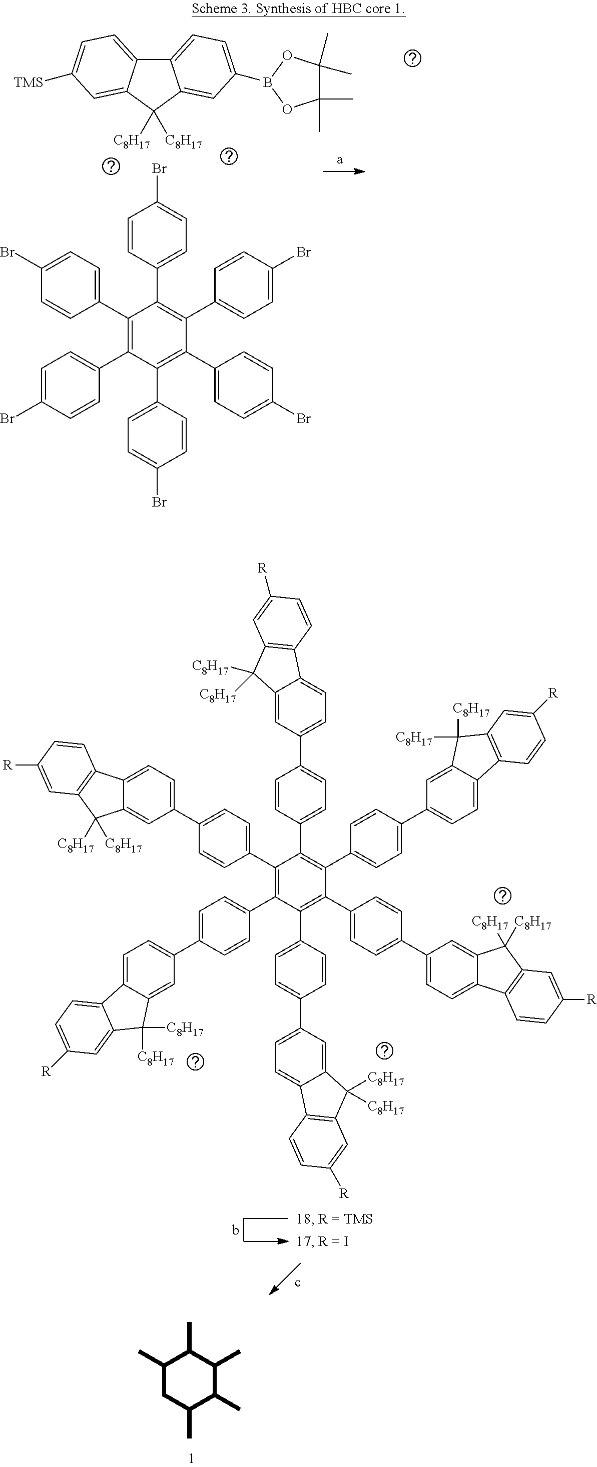

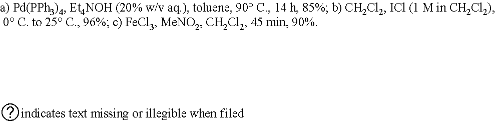

### Example 2

### HBC Core 2 (See Scheme 4)

To a degassed solution of compound 16 (1.5 g, 1 mmol) in CH₂Cl₂ (50 mL) was added FeCl₃ (1 g in 5 mL of MeNO₂). The reaction was allowed to stir for 5 h with argon bubbling through the reaction. Methanol (10 mL) was added and the product was extracted with CH₂Cl₂. A yellow crystalline solid (1 g, 64% yield) was isolated after column chromatography (SiO₂, CH₂Cl₂/pet. spirits 40-60° C. 1:3, R_{f} 0.25) and recrystallisation from CH₂Cl₂. m.p. >250° C.

¹H NMR (500 MHz, CDCl₃): 0.84 (t, J 7, 12H, —CH₃), 0.99 (br, 4H, —CH₂—), 1.08 (br, 4H, —CH₂—), 1.27 (m, 40H, —CH₂—), 2.26 (m, 8H, —CH₂—), 7.06 (t, J 7, 2H, ArH), 7.18 (t, J 7, 2H, ArH), 7.54 (d, J 7, 2H, ArH), 7.62 (m, 4H, ArH), 7.77-7.83 (m, 12H, ArH), 7.89 (m, 2H, ArH), 7.91 (m, 2H, ArH), 7.99 (br s, 2H, ArH). ¹³C NMR (125 MHz, CDCl₃, δ): 14.2, 22.7, 24.2, 29.3 (2), 29.4 (2), 29.5, 30.3, 31.9 (2), 40.6, 55.6, 92.6, 117.3, 117.7, 117.8, 118.1 (2), 119.6 (2), 119.7, 119.8, 119.9, 120.2, 121.2, 121.6, 121.8, 122.5, 124.2, 124.3 (2), 126.5, 127.8 (4), 127.9, 128.0, 128.1 (2), 132.3 (2), 135.9, 136.2, 139.2, 140.6, 141.1, 150.8, 153.5. MS-EI (m/z): M⁺ 1552.6. Elemental analysis: cal. C, 77.41; H, 6.24. found C, 77.19; H, 6.37.

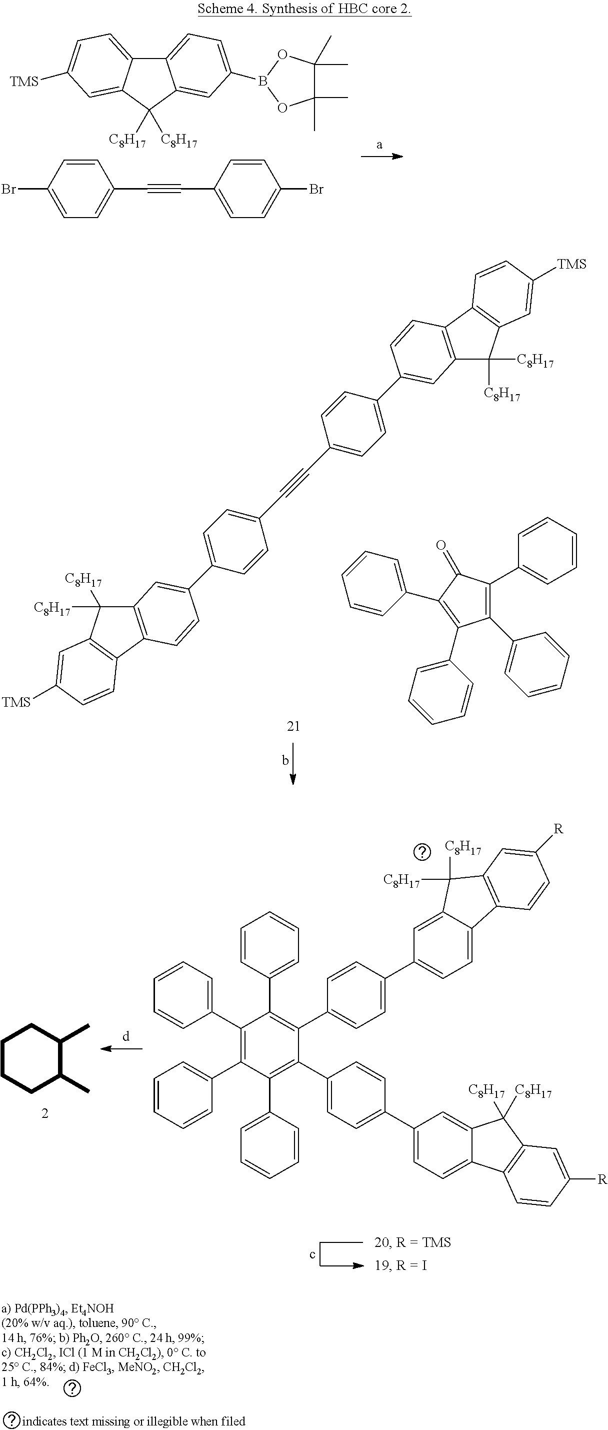

### Example 3

### HBC Core 3 (See Scheme 5)

Compound 19 (2 g, 1.3 mmol) was dissolved in CH₂Cl₂ (500 mL) with argon bubbling through the solution. FeCl₃ (3.8 g, 24 mmol) in nitromethane (20 mL) was added and the solution was stirred at 25° C. for 1 h with argon bubbling through the solution. Methanol (300 mL) was added and the CH₂Cl₂ was removed in vacuo. The precipitate was collected and washed with methanol and petroleum spirits. The residue was dissolved in CH₂Cl₂ and precipitated in diethyl ether. The precipitate was again collected and washed with diethyl ether and petroleum spirits. An orange solid (1.7 g, 83% yield) was obtained after drying in vacuo. m.p. >250° C.

¹H NMR (500 MHz, CDCl₃, δ): 0.86 (t, J 7, 12H, CH₃), 0.98 (br, 8H, CH₂), 1.26 (m, 40H, CH₂), 2.19 (m, 8H, CH₂), 6.99 (m, 4H, ArH), 7.34 (d, J 7, 2H, ArH), 7.53 (d, J 8, 2H, ArH), 7.57 (br, 4H, ArH), 7.63 (d, J 7, 2H, ArH), 7.72 (m, 6H, ArH), 7.87 (m, 4H, ArH), 7.97 (s, 4H, ArH). ¹³C NMR (125 MHz, CDCl₃, δ): 14.2, 22.7, 24.2, 29.4, 29.5, 30.2, 31.9, 40.4, 55.6, 92.6, 118.2, 118.6 (2), 119.9 (4), 120.0 (3), 120.3, 121.6, 121.7, 122.1, 122.9, 124.5, 126.8 (2), 128.2 (2), 128.6, 132.4, 136.1, 136.2, 136.8, 139.2, 140.6, 141.4, 150.9, 153.6. MS-EI (m/z): M⁺ 1552.7. Elemental analysis: cal. C, 77.41; H, 6.24. found C, 63.56; H, 5.97.

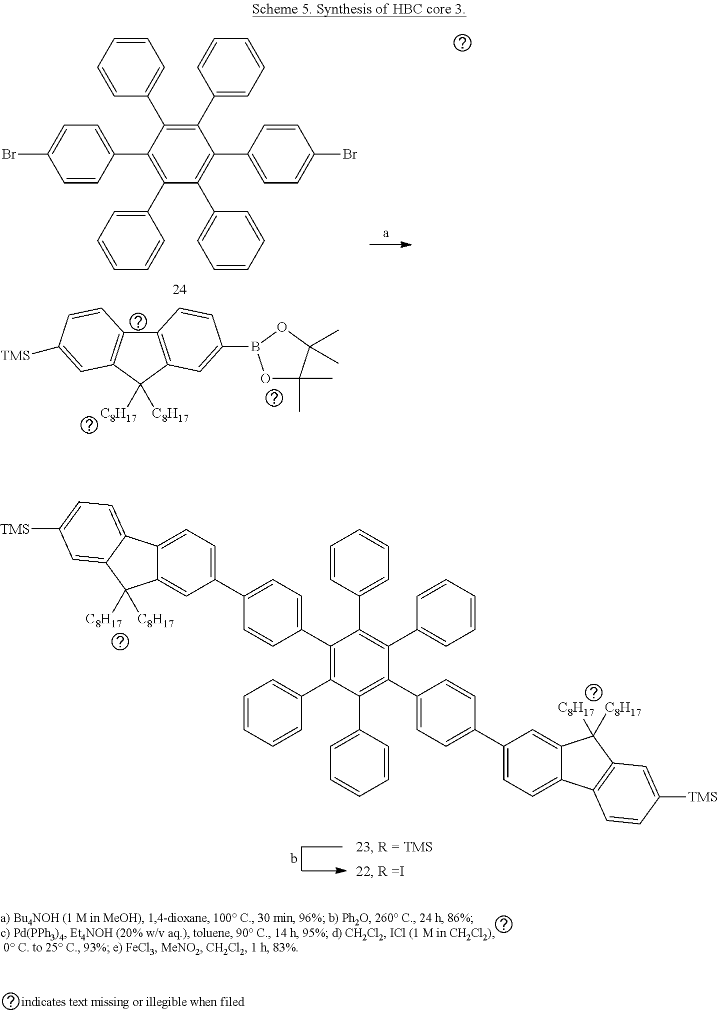

### Example 4

### HBC-Triarylamine Dendrimer 4 (See Scheme 1)

HBC core 1 (0.14 g, 0.04 mmol) and triarylamine oligomer 7 (0.4 g, 0.24 mmol) were placed in a Schlenk tube along with palladium acetate (1 mg) and tri-tert-butylphosphonium tetrafluoroborate (2 mg). Sodium tert-butoxide (50 mg, 0.5 mmol) was transferred into the reaction vessel under an inert atmosphere and toluene (25 mL) was added. The reaction was stirred at 65° C. for 14 h and allowed to cool to 25° C. The mixture was filtered through a plug of silica and a pale yellow solid (0.5 g, 98% yield) was isolated after several precipitations from MeOH. m.p. 165° C.

UV-vis (λ nm, ε 10⁵ M⁻¹ cm⁻¹): 303 (6.4), 329 (7.1), 378 (9.9), 492 (0.3). ¹H NMR (500 MHz, C₆D₆, δ): 0.74 (br m, 108H, —CH₃), 0.93-1.30 (br m, 432H, —CH₂—), 2.07 (br m, 144H, —CH₂— and ArCH₃), 6.90-7.70 (br m, ArH), 7.99 (br, ArH), 8.12 (br, ArH), 8.31 (br, ArH), 9.78 (br, ArH). ¹³C NMR (125 MHz, C₆D₆, δ): 14.2, 19.5, 20.7, 22.2, 22.6, 22.8, 22.9, 24.2, 27.8, 28.9, 29.4, 29.7, 30.0, 30.3, 30.4, 32.0, 32.1, 34.3, 40.8, 40.9, 41.5, 55.6, 120.4, 120.5, 121.2, 121.7, 123.5, 123.6, 124.9, 125.0, 125.1, 125.6, 126.1, 126.7, 127.2, 127.3, 127.4, 128.2, 128.9, 130.3, 132.7, 135.4, 135.5, 140.0, 140.2, 140.3, 140.4, 140.7, 142.0, 143.3, 143.4, 145.6, 147.8, 152.0. MS-MALDI (m/z): M⁺ 12874 (DCTB matrix). Elemental analysis: cal. C, 89.09; H, 8.28; N, 2.63. found C, 88.53; H, 8.17; N, 2.46.

### Example 5

### HBC-Triarylamine Dendrimer 5 (See Scheme 1)

HBC core 2 (0.14 g, 0.09 mmol) and triarylamine oligomer 7 (0.3 g, 0.18 mmol) were placed in a Schlenk tube along with palladium acetate (1 mg) and tri-tert-butylphosphonium tetrafluoroborate (2 mg). Sodium tert-butoxide (50 mg, 0.5 mmol) was transferred into the reaction vessel under an inert atmosphere and toluene (25 mL) was added. The reaction was stirred at 65° C. for 14 h and allowed to cool to 25° C. The mixture was filtered through a plug of silica and a pale yellow solid (0.4 g, 96% yield) was isolated after several precipitations from MeOH. m.p. 152-155° C.

UV-vis: λₘₐₓ=375 nm for thin film on glass. ¹H NMR (500 MHz, C₆D₆, δ): 0.80 (t, J 8, 18H, —CH₃), 0.82 (t, J 8, 6H, —CH₃), 0.94-1.10 (m, 108H, —CH₂—), 1.35-1.51 (m, 48H, —CH₂—), 2.04-2.45 (m, 48H, —CH₂— and tol-CH₃), 6.90-8.66 (br m, 134H, ArH). ¹³C NMR (125 MHz, C₆D₆, δ): 14.1 (2), 14.2, 14.4 (2), 20.7, 20.8, 22.8 (2), 22.9, 23.1 (2), 24.2, 24.3 (2), 28.2, 29.1, 29.2, 29.3, 29.4 (2), 29.5 (3), 29.9 (2), 30.0 (2), 30.1, 30.3, 30.4, 30.5, 30.9, 32.0 (2), 32.3 (2), 40.8, 41.0, 55.6 (3), 120.4, 120.5 (2), 121.2, 121.7, 123.5, 123.6, 123.8, 125.6 (2), 126.1, 126.2, 126.7, 127.3, 127.5 (2), 127.6 (2), 127.7, 127.8, 127.9, 128.0, 128.1, 128.3 (2), 128.4, 129.0, 130.2, 130.3 (2), 130.4, 132.8, 135.5 (2), 140.1, 140.3 (2), 140.5, 140.7, 142.1, 143.3, 143.4, 145.6, 147.8 (2), 152.0 (2). MS-MALDI (m/z): M⁺ 4607.8. Elemental analysis: cal. C, 89.65; H, 7.92; N, 2.43. found C, 89.71; H, 7.93; N, 2.40.

### Example 6

### HBC-Triarylamine Dendrimer 6 (See Scheme 1)

HBC core 3 (0.07 g, 0.045 mmol) and triarylamine oligomer 7 (0.15 g, 0.09 mmol) were placed in a Schlenk tube along with palladium acetate (1 mg) and tri-tert-butylphosphonium tetrafluoroborate (2 mg). Sodium tert-butoxide (30 mg, 0.5 mmol) was transferred into the reaction vessel under an inert atmosphere and toluene (20 mL) was added. The reaction was stirred at 65° C. for 14 h and allowed to cool to 25° C. The mixture was filtered through a plug of silica and a pale yellow solid (0.2 g, 96% yield) was isolated after several precipitations from MeOH. m.p. 151-153° C.

UV-vis: λₘₐₓ=377 and 440 (sh) nm for thin film on glass. ¹H NMR (500 MHz, C₆D₆, δ): 0.77 (t, J 8, 18H, —CH₃), 0.79 (t, J 8, 6H, —CH₃), 0.91-1.13 (m, 108H, —CH₂—), 1.33 (br m, 48H, —CH₂—), 2.01-2.24 (m, 48H, —CH₂— and tol-CH₃), 6.88-8.53 (br m, 134H, ArH). ¹³C NMR (125 MHz, C₆D₆, δ): 14.3, 14.4, 14.5, 20.8, 23.0 (2), 23.2, 24.4, 29.5, 29.6, 29.7, 30.0, 30.1, 30.5, 30.6, 30.9, 32.1, 32.2, 32.4, 41.0, 55.7, 120.5, 120.6, 120.7, 121.3, 121.8, 123.7, 125.2, 125.4, 125.7, 125.8, 126.3, 127.4, 127.7 (2), 127.8, 127.9, 128.0, 128.3, 128.5, 129.1, 130.4, 130.5, 132.9, 135.6, 140.2, 140.4, 140.6, 140.8, 140.9, 142.2, 143.5, 145.8, 148.0, 152.1, 152.2. MS-MALDI (m/z): M⁺ 4607.8. Elemental analysis: cal. C, 89.65; H, 7.92; N, 2.43. found C, 89.63; H, 7.92; N, 2.45.

### Example 7

### Triarylamine Oligomer 7 (See Scheme 6)

Compound 25 (0.25 g, 0.14 mmol) was heated under vacuum at 200° C. for 4 h. The reaction was cooled and dissolved in CH₂Cl₂. A pale yellow solid (0.23 g, 98% yield) was isolated after several precipitations from MeOH. m.p. 103-104° C.

¹H NMR (500 MHz, C₆D₆, δ): 0.74 (m, 12H, —CH₃), 0.93-1.10 (m, 48H, —CH₂—), 2.07 (m, 20H, —CH₂—), 4.89 (s, 1H, ArNH), 6.73-6.81 (m, 4H, ArH), 6.92 (m, 8H, ArH), 7.12-7.26 (m, 21H, ArH), 7.54-7.73 (m, 20H, ArH). ¹³C NMR (125 MHz, C₆D₆, δ): 14.1, 20.6, 22.8, 24.2, 29.3, 29.4, 30.3, 30.4, 32.0, 40.8, 40.9, 55.6, 118.6, 120.4, 121.2, 121.7, 122.8, 123.5, 124.4, 125.0, 125.6, 126.1, 126.6, 126.8, 128.9, 130.0, 130.1, 130.2, 132.7, 135.4, 140.0, 140.2, 140.4, 140.7, 142.0, 143.3, 143.4, 145.6, 147.7, 152.0. MS-EI (m/z): 828.5 (M²⁺), 1655.0 (M⁺). Elemental analysis: cal. C, 88.46; H, 8.15; N, 3.38. found C, 88.43; H, 8.16; N, 3.36.

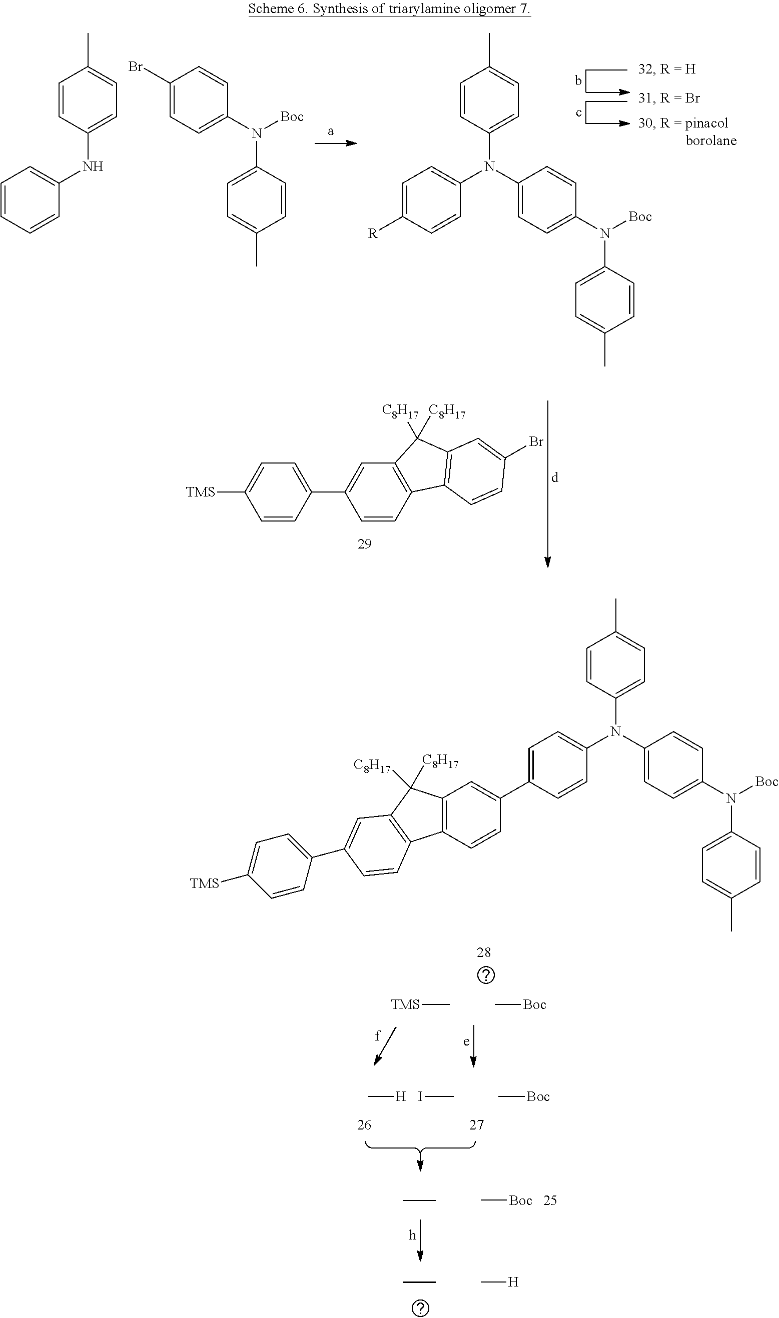

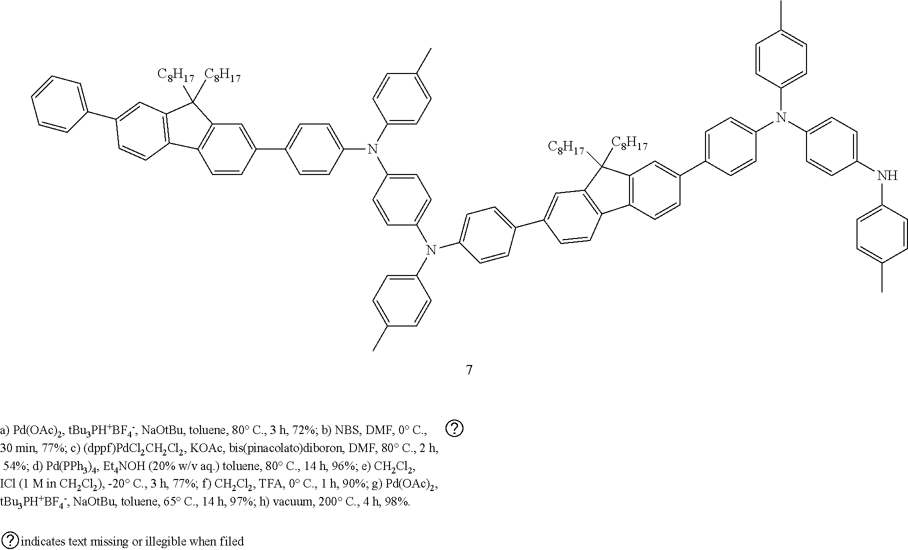

### Example 8

### HBC Precursor 17

Compound 18 (1 g, 0.3 mmol) was dissolved in CHCl₃ (25 mL) and cooled to 0° C. Iodine monochloride (5 mL, 1 M in CH₂Cl₂) was added dropwise and the reaction was allowed to stir at 0° C. br 30 min. Sodium thiosulfate (20 mL, 1 M aq.) was added and the organic layer was collected and washed with brine. The solvent was removed in vacuo and the residue was purified by column chromatography (SiO₂, pet. spirit 40-60/CH₂Cl₂ 2:1, R_{f} 0.8) to give a yellow powder (1.05 g, 96% yield). m.p. 140-141° C.

¹H NMR (500 MHz, CDCl₃, 8): 0.67 (br m, 24H, —CH₂—), 0.79 (br m, 36H, —CH₃), 1.04-1.20 (br m, 120H, —CH₂—), 1.92 (br m, 24H, —CH₂—), 7.07 (d, J 8, 12H, ArH), 7.30 (d, J 8, 12H, ArH), 7.37-7.46 (br m, 18H, ArH), 7.64 (br m, 18H, ArH). ¹³C NMR (125 MHz, CDCl₃, δ): 14.3, 22.9, 23.9, 29.4, 30.2, 31.1, 32.0, 40.5, 55.6, 92.6, 120.1, 121.2, 121.6, 125.9, 126.3, 132.3, 136.1, 139.5, 140.6, 151.0, 153.5. MS-MALDI (m/z): M⁺ 3621.5. Elemental analysis: cal. C, 71.63; H, 7.35. found C, 72.24; H, 7.14.

### Example 9

### HBC Precursor 18

Hexakis(4-bromophenyl)benzene (0.5 g, 0.5 mmol), 2-pinacolborolane-7-trimethylsilyl-9,9-dioctylfluorene (1.9 g, 3.25 mmol) and tetrakis (triphenylphosphine)palladium (23 mg, 0.02 mmol) was dissolved in degassed toluene (20 mL) under N₂. Degassed Et₄NOH (10 mL, 20% in H₂O) was added and the reaction was heated at 100° C. for 14 h under N₂. The reaction mixture was poured into methanol (100 mL) and the resulting precipitate was collected. The residue was purified by column chromatography (SiO₂, pet. spirit 40-60/CH₂Cl₂ 2:1, R_{f} 0.9) and a white powder (1.4 g, 85% yield) was isolated. m.p. 154° C.

¹H NMR (500 MHz, CDCl₃, δ): 0.37 (s, 54H, TMS), 0.75 (br m, 24H, —CH₂—), 0.85 (m, 36H, —CH₃), 1.11-1.27 (m, 120H, —CH₂—), 2.01 (m, 24H, —CH₂—), 7.13 (d, J 9, 12H, ArH), 7.37 (d, J 9, 12 H, ArH), 7.44 (d, J 7, 6H, ArH), 7.51 (m, 18H, ArH), 7.70 (d, J 8, 12H, ArH). ¹³C NMR (125 MHz, CDCl₃, δ): −0.6, 14.4, 22.9, 24.0, 29.4, 30.2, 32.1, 40.5, 55.3, 119.2, 120.1, 121.4, 125.6, 126.0, 126.2, 127.5, 127.9, 128.5, 129.0, 129.3, 132.0, 132.1, 132.4, 138.9, 139.1, 140.0, 140.3, 140.5, 140.7, 141.7, 150.3, 151.9. MS-MALDI (m/z): M⁺ 3299.4. Elemental analysis: cal. C, 85.18; H, 9.71. found C, 85.20; H, 9.74.

### Example 10

### Compound 19

To a solution of compound 20 (2 g, 1.4 mmol) in CH₂Cl₂ (50 mL) at 0° C. was added iodine monochloride (1 M in CH₂Cl₂, 5 mL). The reaction was allowed to stir for 1 h and warmed to 25° C. Sodium thiosulfate (1 M aq., 50 mL) was added and the product was extracted with CH₂Cl₂. A white solid (1.8 g, 84% yield) was isolated after column chromatography (SiO₂, CH₂Cl₂/pet. spirits 40-60° C. 1:3, R_{f} 0.4). m.p. 112-115° C.

¹H NMR (500 MHz, CDCl₃, δ): 0.64 (br, 8H, —CH₂—), 0.85 (t, J 7, 12H, —CH₃), 1.06-1.22 (m, 40H, —CH₂—), 1.94 (m, 8H, —CH₂—), 6.90 (m, 20H, ArH), 7.04 (d, J 8, 4H, ArH), 7.27 (d, J 8, 4H, ArH), 7.42 (m, 6H, ArH), 7.65 (m, 6H, ArH). ¹³C NMR (125 MHz, CDCl₃, δ): 14.1, 22.6, 23.6, 29.1, 29.9, 31.7, 40.1, 55.3, 92.3, 119.8, 120.9, 121.3, 125.2, 125.4, 125.9, 126.6, 126.7, 131.4, 131.5, 132.0, 135.8, 138.0, 139.0, 139.7, 139.8, 140.4, 140.6, 140.7, 150.7, 153.6. MS-EI (m/z): M⁺ 1564.6. Elemental analysis: cal. C, 76.81; H, 6.96. found C, 76.81; H, 7.00.

### Example 11

### Compound 20

This procedure was adapted from the literature [Hill, J. P.; Jin, W.; Kosaka, A.; Fukushima, T.; Ichihara, H.; Shimomura, T.; Ito, K.; Hashizume, T.; Ishii, N.; Aida, T. Science 2004, 304, 1481]. Compound 21 (1.4 g, 1.2 mmol) and tetraphenylcyclopentadienone (0.5 g, 1.3 mmol) were dissolved in diphenyl ether (2 mL) and heated at 260° C. for 24 h. A white solid (2 g, 99% yield) was isolated after column chromatography (SiO₂, CH₂Cl₂/pet. spirits 40-60° C. 1:3, R_{f} 0.5). m.p. 98-99° C.

¹H NMR (500 MHz, CDCl₃, δ): 0.29 (s, 18H, TMS), 0.63 (br, 8H, —CH₂—), 0.77 (t, J 7, 12H, —CH₃), 1.01-1.18 (m, 40H, —CH₂—), 1.91 (m, 8H, —CH₂—), 6.84 (m, 20H, ArH), 7.00 (m, 4H, ArH), 7.23 (m, 4H, ArH), 7.30-7.45 (m, 8H, ArH), 7.62 (m, 4H, ArH). ¹³C NMR (125 MHz, CDCl₃, δ): −0.9, 14.1, 22.6, 23.6, 29.0, 29.1, 29.9, 31.7, 40.0, 55.0, 118.9, 119.8, 121.0, 123.2, 125.2, 125.4, 125.7, 126.6, 126.7, 127.6, 129.7, 131.4, 131.5, 131.7, 131.9, 138.2, 138.7, 139.5, 139.8, 139.9, 140.0, 140.6, 140.7, 141.4, 150.0, 151.5, 157.2. MS-EI (m/z): M⁺ 1457.7. Elemental analysis: cal. C, 87.42; H, 8.72. found C, 87.50; H, 8.66.

### Example 12

### Compound 21

2-Pinacolborolane-7-trimethylsilyl-9,9-dioctylfluorene (3.5 g, 6 mmol), 4,4′-dibromophenylacetylene (1 g, 3 mmol) and Pd(PPh₃)₄ (50 mg) were dissolved in degassed toluene (30 mL). Tetraethylammonium hydroxide solution (20% wt. in water, 10 mL) was thoroughly degassed and added to the reaction mixture. The resulting solution was heated at 90° C. for 14 h and the product was extracted into toluene. The toluene solution was dried over MgSO₄ and filtered through a plug of silica. A pale yellow solid (2.5 g, 76% yield) was obtained after column chromatography (SiO₂, Pet. Spirits/CH₂Cl₂ 3:1, R_{f} 0.6) and precipitation from methanol. White crystals were obtained from recrystallisation with isopropanol for analysis. m.p. 171° C.

¹H NMR (500 MHz, CDCl₃, δ): 0.36 (s, 18H, TMS), 0.75 (br, 8H, —CH₂—), 0.83 (t, J 7, 12H, —CH₃), 1.10 (br m, 40H, —CH₂—), 2.04 (m, 8H, —CH₂—), 7.52-7.80 (m, 20H, ArH). ¹³C NMR (125 MHz, CDCl₃, δ): −0.9, 14.1, 22.6, 23.8, 29.1, 29.2, 29.9, 31.8, 40.2, 55.1, 90.2, 119.1, 120.1, 121.3, 122.0, 125.9, 127.0, 127.6, 131.9, 132.0, 139.2, 140.8, 141.2, 141.5, 150.2, 151.7. MS-EI (m/z): M⁺ 1099.3. Elemental analysis: cal. C, 85.18; H, 9.71. found C, 85.31; H, 9.74.

### Example 13

### Compound 22

Compound 23 (2 g, 1.4 mmol) was dissolved in CH₂Cl₂ (25 mL) and cooled to 0° C. Iodine monochloride solution (1 M in CH₂Cl₂, 3 mL) was added dropwise and the reaction was stirred at 0° C. for 1 h. Sodium thiosulfate solution (1 M) was added and the reaction stirred vigorous for 30 min. The organic phase was collected, dried over MgSO₄ and filtered through a plug of silica. A white solid (2 g, 93% yield) was obtained after precipitation from methanol. m.p. 120° C.

¹H NMR (500 MHz, CDCl₃, 8): 0.61 (br, 8H, CH₂), 0.83 (t, J 7, 12H, CH₃), 1.04-1.21 (m, 40H, CH₂), 1.90 (m, 8H, CH₂), 6.91 (m, 20H, ArH), 6.94 (d, J 9, 4H, ArH), 7.20 (d, J 9, 4H, ArH), 7.41 (m, 6H, ArH), 7.65 (m, 6H, ArH). ¹³C NMR (125 MHz, CDCl₃, δ): 14.1, 22.6, 23.6, 29.1, 29.2, 29.9, 31.7, 40.2, 55.3, 92.3, 119.8, 120.9, 121.3, 125.2, 125.9, 126.7, 131.5, 131.9, 132.0, 135.8, 137.8, 139.0, 140.3, 140.4, 140.5, 140.6, 150.6, 153.4. MS-EI (m/z): M⁺ 1564.7. Elemental analysis: cal. C, 76.81; H, 6.96. found C, 74.42; H, 5.98.

### Example 14

### Compound 23

Compound 24 (1 g, 1.44 mmol), 2-pinacolborolane-7-trimethylsilyl-9,9-dioctylfluorene (1.76 g, 3 mmol) and Pd(PPh₃)₄ (40 mg) were dissolved in degassed toluene (15 mL). Tetraethylammonium hydroxide solution (20% wt. in water, 5 mL) was thoroughly degassed and added to the reaction mixture. The resulting solution was heated at 90° C. for 14 h and the product was extracted into toluene. The toluene solution was dried over MgSO₄ and filtered through a plug of silica. A white solid (2 g, 95% yield) was obtained after precipitation from methanol. m.p. 163-165° C.

¹H NMR (500 MHz, CDCl₃, δ): 0.33 (s, 18H, TMS), 0.68 (br, 8H, CH₂), 0.84 (t, J 7, 12H, CH₃), 1.07-1.22 (m, 40H, CH₂), 1.95 (m, 8H, CH₂), 6.91 (m, 20H, ArH), 6.96 (d, J 9, 4H, ArH), 7.23 (d, J 9, 4H, ArH), 7.43 (m, 4H, ArH), 7.47 (m, 4H, ArH), 7.67 (m, 4H, ArH). ¹³C NMR (125 MHz, CDCl₃, δ): −0.9, 14.1, 22.6, 23.7, 29.0, 29.1, 29.9, 31.7, 40.1, 55.0, 118.9, 119.8, 121.0, 125.2, 125.6, 126.7, 127.6, 131.5, 131.7, 131.8, 138.0, 138.8, 139.6, 139.8, 140.0, 140.7, 141.4, 150.1, 151.5. MS-EI (m/z): M⁺ 1457.8. Elemental analysis: cal. C, 87.42; H, 8.72. found C, 87.38; H, 8.70.

### Example 15

### Triarylamine Oligomer 25

Compound 26 (0.19 g, 0.24 mmol) and compound 27 (0.25 g, 0.24 mmol) were placed in a Schlenk tube along with palladium acetate (1 mg) and tri-tert-butylphosphonium tetrafluoroborate (2 mg). Sodium tert-butoxide (50 mg, 0.5 mmol) was transfered into the reaction vessel under an inert atmosphere and toluene (25 mL) was added. The reaction was stirred at 65° C. for 14 h and allowed to cool to 25° C. The mixture was filtered through a plug of silica and a plae yellow solid (0.4 g, 97% yield) was isolated after several precipitations from MeOH. m.p. 101° C.

¹H NMR (500 MHz, C₆D₆, δ): 0.79 (t, J 7, 6H, —CH₃), 0.80 (t, J 7, 6H, —CH₃), 0.97-1.14 (m, 48H, —CH₂—), 1.42 (s, 9H, Boc), 2.04 (s, 3H, Tol), 2.10 (5, 3H, Tol), 2.12 (s, 6H, Tol), 2.15 (m, 8H, —CH₂—), 6.90 (d, J 7, 4H ArH), 6.95 (d, J 8, 4H, ArH), 7.08-7.31 (m, 24H, ArH), 7.52-7.59 (m, 10H ArH), 7.64-7.70 (m, 6H, ArH), 7.76 (d, J 7, 4H, ArH). ¹³C NMR (125 MHz, C₆D₆, δ): 14.3, 15.6, 20.8, 23.0, 24.4, 28.3, 29.5, 29.6, 30.5, 32.1, 41.0, 55.7, 80.3, 120.6, 120.7, 121.3, 121.8, 123.7, 123.9, 124.2, 125.2, 125.5, 125.7, 126.3, 126.8, 127.4, 127.9, 129.1, 129.6, 129.9, 130.4, 132.9, 133.1, 135.2, 135.6, 135.7, 136.0, 138.6, 140.2, 140.3, 140.4, 140.6, 140.9, 141.5, 142.2, 143.4, 143.5, 145.6, 145.7, 147.6, 147.9, 148.0, 152.1, 153.8. Elemental analysis: cal. C, 86.84; H, 8.15; N, 3.19. found C, 86.82; H, 8.20; N, 3.20.

### Example 16

### Triarylamine Monomer 26

Compound 28 (2 g, 2 mmol) was dissolved in CH₂Cl₂ (10 mL) and the solution was cooled to 0° C. Trifluoroacetic acid was added dropwise under N₂ and the reaction was allowed to stir for 1 h at 0° C. A solution of sodium hydrogen carbonate was added to the reaction and the product was extracted into CH₂Cl₂ (50 mL). A pale yellow solid (1.5 g, 90% yield) was obtained after purification by column chromatography (SiO₂, pet. spirit/CH₂Cl₂ 1:1, R_{f} 0.7). m.p. 71° C.

¹H NMR (500 MHz, C₆D₆, δ): 0.78 (t, J 8, 6H, —CH₂CH₃), 0.93-1.13 (m, 24H, —CH₂—), 2.10 (m, 10H, —CH₂— and ArCH₃), 6.94 (br m, 4H, ArH), 7.22 (m, 4H, ArH), 7.25 (m, 4H, ArH), 7.52 (m, 4H, ArH), 7.64 (m, 5H, ArH), 7.73 (d, J 8, 2H, ArH). ¹³C NMR (125 MHz, C₆D₆, δ): 14.2, 20.7, 22.8, 24.3, 29.4, 29.5, 30.4, 32.0, 40.8, 55.6, 118.6, 120.4, 120.6, 121.2, 121.8, 122.8, 124.5, 126.2, 126.7, 127.0, 130.0, 130.2, 132.2, 134.1, 134.8, 138.8, 139.9, 140.7, 140.9, 142.6, 152.1. MS-EI (m/z): M⁺ 828.5. Elemental analysis: cal. C, 88.36; H, 8.27; N, 3.38. found C, 89.51; H, 8.48; N, 3.02.

### Example 17

### tert-butyl 4-((4-(7-(4-iodophenyl)-9,9-dioctyl-9H-fluoren-2-yl)phenyl)(p-tolyl)amino)phenyl(p-tolyl)carbamate 27

To a solution of 28 (4.163 g, 4.16 mmol) dissolved in dry degassed CH₂Cl₂ (150 mL) at −20° C. was added ICl (14.6 mL, 1.0M in CH₂Cl₂, 14.6 mmol) dropwise over 20 minutes. The solution went a dark green. The reaction mix was stirred at −20° C. for 3 hours then and excess of Et₃N (5 mL) added. The reaction mix was deactivated by addition of an excess of saturated sodium thiosulphate solution. The organic phase was separated and the reaction mix extracted with CH₂Cl₂ (2×50 mL). The combined organic phase was washed with brine and dried over MgSO₄. The solvent was removed under vacuum. The product was purified by column chromatography, toluene R_{f}=0.43, then by dissolving in a minimum of ether and adding the ether solution dropwise to 250 mL of methanol at −20° C. The product was collected by filtration, washed with cold methanol and dried under a stream of air then under vacuum overnight (3.20 g, 77%).

NMR: δ_{H} 7.751 (d, 1H J=1.0 Hz, Ar—H), 7.667 (dd, 2H J=7.9 & 7.9 Hz, Ar—H), 7.611 (d, 1H J=1.2 Hz, Ar—H), 7.57-7.53 (m, 3H, Ar—H), 7.521 (d, 2H J=8.6 Hz, Ar—H), 7.384 (dd, 1H J=7.9 & 1.0 Hz, Ar—H), 7.237 (dd, 4H J=8.1 & 8.1 Hz, Ar—H), 7.196 (d, 2H J=8.6 Hz, Ar—H), 7.12-7.05 (m, 6H, Ar—H), 6.908 (dd, 4H J=8.2 & 1.4 Hz, Ar—H overlapping solvent peak), 2.116 (brm, 4H, octyl-a-CH₂s) overlapping 2.074 (s, 3H, Me), 2.015 (s, 3H, Me), 1.396 (s, 9H, BOC-Me₃), 1.119-0.893 (brm, 24H, octyl-CH₂'s), 0.760 (t, 6H J=7.1 Hz, octyl-Me's). m/z: 1055 (M₊, 30%), 998 (45%), 954 (M-BOC₊, 100%). IR: CO 1706 cm⁻¹. Elemental Analysis: Calculated for C₆₆H₇₅IN₂O₂ C, 75.12; H, 7.16; I, 12.03; N, 2.65; O, 3.03. Found C, 75.26; H, 7.16; N, 2.83.

### Example 18

### tert-butyl 4-((4-(9,9-dioctyl-7-(4-(trimethylsilyl)phenyl)-9H-fluoren-2-yl)phenyl)(p-tolyl)amino)phenyl(p-tolyl)carbamate 28

The product was generated by a Suzuki-Miyura reaction. The reagents 29 (7.0 g, 11.33 mmol) and 30 (6.69 g, 11.33 mmol) were placed in a 250 ml RB flask with toluene (100 mL) and Et₄NOH (40 mL, 20 Wt %). The combined reaction mix degassed by bubbling N₂ through it for 30 minutes. The catalyst Pd(PPh₃)₄ (261 mg, 0.226 mmol) was added and the reaction mix degassed for a further 10 minutes. The reaction mix was then heated to 80° C. for 16 hours, cooled to ambient temperature and the aqueous phase decanted. The toluene solution was filtered through a pad of silica and the silica washed with toluene. The crude product was recovered by removal of the solvent under vacuum and purified by column chromatography (20 cm×8 cm) using toluene. R_{f}=0.49 (10.92 g, 96.2%). Analytically pure material was recovered by dissolving the product in a minimum amount of ether and adding dropwise to 250 mL of rapidly stirred methanol at 0° C. The product was recovered by filtration and washed with cold methanol, dries under a stream of air and then overnight under high vacuum.

NMR: δ_{H} 7.79 (1H, d J=1 Hz, Ar—H), 7.73 (1H, d J=1 Hz, Ar—H), 7.68-7.70 (3H, m, Ar—H), 7.66 (1H, d J=7.5 Hz, Ar—H), 7.61 (1H, dd J=7.5 & 2.0 Hz, Ar—H), 7.53-7.57 (3H, m, Ar—H), 7.50 (2H, d J=8.5 Hz, Ar—H), 7.19-7.23 (4H, m, Ar—H), 7.17 (2H, d J=8.5 Hz, Ar—H), 7.09 (2H, d J=8.5 Hz, Ar—H), 7.06 (2H, d J=8.5 Hz, Ar—H), 6.88 (4H, brd J=7.0 Hz, Ar—H), 2.14 (4H, m, α-CH₂), 2.08 (3H, s, tolyl-CH₃), 2.02 (3H, s, tolyl-CH₃), 2.08 (9H, s, BOC—(CH₃)₃), 0.94-1.12 (24H, m, octyl-CH₂'s), 0.77 (6H, t J=7.5 Hz, octyl-CH₃), 0.25 (9H, s, TMS-(CH₃)₃). δ_{C} 153.8, 152.18, 152.15, 147.7, 145.5, 142.6, 141.4, 140.91, 140.86, 140.5, 140.2, 139.0, 138.6, 35.9, 135.2, 134.2 (Ar—H), 133.1, 130.4 (Ar—H), 129.6 (Ar—H), 128.4 (Ar—H), 128.0 (Ar—H), 127.4 (Ar—H), 127.1 (Ar—H), 126.8 (Ar—H), 126.3 (Ar—H), 125.5 (Ar—H), 124.2 (Ar—H), 123.9 (Ar—H), 121.9 (Ar—H), 121.3 (Ar—H), 120.7 (Ar—H), 120.6 (Ar—H), 80.3, 55.7, 40.9 (CH₂), 32.1 (CH₂), 30.4 (CH₂), 29.6 (CH₂), 29.5 (CH₂), 28.3 (BOC—(CH₃)₃), 24.4 (CH₂), 22.9 (CH₂), 20.83 (CH₃), 20.79 (CH₃), 14.3 (Si—(CH₃)₃). EI m/z 1000.5 (M₊-H, 4%), 900.4 (M⁺-BOC, 100%). Elemental analysis: Calculated for C₆₉H₈₄N₂O₂SiC, 82.75; H, 8.45; N, 2.80. Found C, 82.96; H, 8.27; N, 3.01.

### Example 19

### (4-(7-bromo-9,9-dioctyl-9H-fluoren-2-yl)phenyl)trimethylsilane 29

The product was generated by a statistical Suzuki-Miyura reaction. The reagents TMS-C₆H₄-Borolane (5.0 g, 18.1 mmole) and Br₂F8 (15.9 g, 27.0 mmoles) were placed in a 250 ml RB flask with toluene (100 mls) and Et₄NOH (40 mls, 20 Wt %). The combined reaction mix degassed by bubbling N₂ through it for 30 minutes. The catalyst Pd(PPh₃)₄ (0.416 g, 0.36 mmole) was added and the reaction mix degassed for a further 10 minutes. The reaction mix was then heated to 80° C. for 16 hours, cooled to ambient temperature and the aqueous phase decanted. The toluene solution was filtered through a pad of silica and the silica washed with toluene. The crude product was recovered by removal of the solvent under vacuum and purified by column chromatography (20 cm×8 cm) using petroleum ether (40-60). R_{f}: 0.34 (7.35 g, 65%).

¹H-NMR (500 MHz, C₆D₆): δ_{H} 7.739 (1H, d J=7.5 Hz, Ar—H), 7.64-7.69 (4H, m, Ar—H), 7.604 (1H, dd J=8.5 & 1.5 Hz, Ar—H), 7.590 (1H, d J=7.5 Hz, Ar—H), 7.557 (1H, d J=1.5 Hz, Ar—H), 7.495 (1H, d J=1.5 Hz, Ar—H), 7.482 (1H, dd J=8.5 & 1.5 Hz, Ar—H), 1.96-2.02 (4H, m, α-CH₂), 1.05-1.25 (20H, m, octyl-CH₂'s), 0.836 (6H, t J=7.0 Hz, octyl-CH₃), 0.674 (4H, m, octyl-CH₂), 0.342 (9H, s, Si—(CH₃)₃). ¹³C NMR (125 MHz, C₆D₆): δ_{C} 153.5, 151.2, 142.1, 140.7, 140.0, 139.63, 139.54, 134.1 (Ar—H), 130.2 (Ar—H), 126.8 (Ar—H), 126.41 (Ar—H), 126.38 (Ar—H), 121.8 (Ar—H), 121.34, 121.27 (Ar—H), 120.28 (Ar—H), 55.7, 40.5 (α-CH₂), 32.0 (CH₂), 30.2 (CH₂), 29.46 (CH₂), 29.42 (CH₂), 24.0 (CH₂), 22.9 (CH₂), 14.3 (CH₃), −0.82 (Si—(CH₃)₃). m.p.: 81-82° C. EI m/z 616.4 (M⁺, 95%), 618.4 (M⁺, 100%), 389.1 (M⁺-octyl₂H). Elemental Analysis: Calculated for C₃₈H₅₃BrSi C, 73.87; H, 8.65. Found C, 73.77; H, 8.73.

### Example 20

### tert-butyl 4-((4-(4,4,5,5-tetramethyl-1,3,2-dioxaborolan-2-yl)phenyl)(p-tolyl)amino)phenyl(p-tolyl)carbamate 30

A 250 ml RB flask was loaded with tert-butyl 4-((4-bromophenyl)(p-tolyl)amino)phenyl(p-tolyl)carbamate 31 (12.2 g, 22.45 mmol), bis(pinacolato)diboron (8.6 g, 33.67 mmol), KOAc (6.6 g, 67.0 mmol) and (dppf)PdCl₂.CH₂Cl₂ (0.459 g, 0.56 mmol) and placed under nitrogen. Dry degassed DMF (90 mL) was added and the reaction mix heated to 80° C. for 2 hours. To the cooled reaction mix was added H₂O (300 mL) and the reaction mix extracted with toluene, 3×70 mL. The combined toluene extracts were washed with H2O, 3×50 mL and dried over MgSO₄. The volume of the filtrate was reduced to approx 50 mL and the solution filtered through a pad of silica and the silica washed with toluene. The solvent was removed form the filtrate to leave a crude product. The product was purified by column chromatography using CH₂Cl₂ as solvent. R_{f}=0.39 (CH₂Cl₂). Analytically pure material was recovered by recrystallisation from IPA (12.19 g, 54%).

NMR: δ_{H} 8.009 (2H, d J=8.5 Hz, Ar—H), 7.154 (2H, d J=8.5 Hz, Ar—H), 7.125 (2H, d J=8.5 Hz, Ar—H), 7.103 (2H, d J=8.5 Hz, Ar—H), 6.986 (2H, d J=8.5 Hz, Ar—H), 6.944 (2H, d J=8.5 Hz, Ar—H), 6.854 (2H, d J=8.5 Hz, Ar—H), 6.791 (2H, d J=8.5 Hz, Ar—H), 2.021 (3H, s, tolyl-CH₃), 2.001 (3H, s, tolyl-CH₃), 1.363 (9H, s, BOC—(CH₃)₃), 1.100 (12H, s, pinocolato-(CH₃)₄). δ_{C} 153.7, 151.2, 145.22, 145.16, 138.9, 136.7 (Ar—H), 135.2, 133.3, 130.3 (Ar—H), 129.6 (Ar—H), 128.3, 127.9 (Ar—H), 127.4 (Ar—H), 125.9 (Ar—H), 124.5 (Ar—H), 122.1 (Ar—H), 83.5, 80.3, 28.3 (CH₃), 24.9 (CH₃), 20.82 (CH₃), 20.77 (CH₃). EI m/z 590.4 (M⁺, 6%), 490.3 (M⁺-BOC, 100%). Elemental analysis: Calculated for C₃₇H₄₃BN₂O₄ C, 75.25; H, 7.34; N, 4.74. Found C, 75.24; H, 7.40; N, 4.74.

### Example 21

### tert-butyl 4-((4-bromophenyl)(p-tolyl)amino)phenyl(p-tolyl)carbamate 31

Under N₂, a solution of NBS (1.94 g, 10.87 mmol) in dry, degassed DMF (10 mL), was added dropwise over 30 minutes to a solution of tert-butyl 4-(phenyl(p-tolyl)amino)phenyl(p-tolyl)carbamate 32 (5.0 g, 10.87 mmol) DMF (20 mL) under N₂ at 0° C. The reaction mix was allowed to stir at 0° C. for two hours then the reaction deactivated by addition of H₂O (50 mL). The product was recovered by extraction with EtOAc, 3×20 mL, the combined extracts washed with H₂O, brine and then dried over MgSO₄. The solvent was removed under vacuum and the product purified by column chromatography. R_{f}=0.29 (1:1 CH₂Cl₂:Petroleum Ether). Recrystallisation from petroleum ether generated analytically pure material as a white solid (4.57 g, 77%).

NMR: δ_{H} 7.189 (d, 4H J=7.7 Hz, Ar—H), 7.093 (d, 2H J=8.8 Hz, Ar—H), 6.92-6.87 (m, 6H, Ar—H), 6.705 (d, 2H J=8.8 Hz, Ar—H), 2.058 (s, 3H, Ar-Me), 2.024 (s, 3H, Ar-Me), 1.397 (s, 9H, BOC-Me₃). IR: ν CO (neat) 1705 cm⁻¹. Elemental analysis: Calculated for C₃₁H₃₁BrN₂O₂ C, 68.51; H, 5.74; N, 5.15. Found C, 68.58; H, 5.94; N, 5.17.

### Example 22

### tert-butyl 4-(phenyl(p-tolyl)amino)phenyl(p-tolyl)carbamate 32

Buchwald-Hartwig reaction standard conditions. Compounds N-4-bromophenyl-tolylaniline (6.36 g, 34.7 mmol), tert-butyl 4-bromophenyl(p-tolyl)carbamate (12.57 g, 34.7 mmol) and Pd(OAc)₂ (0.156 g, 0.69 mmol) were placed in a 500 ml RB flask and pumped into a glove-box where NaO^{t}Bu (5.0 g, 52 mmol) was added. A suba seal was placed into the flask, which was removed from the glove-box and the flask charged with 200 mL of dry, degassed toluene. Finally ^{t}Bu₃PH⁺BF₄⁻ (0.200 g, 0.69 mmol) was added under a counter flow of N₂. The reaction mix was then heated to 80° C. for 3 hous. The reaction mixture was deactivated by slow addition of NH₄Cl (5 g, excess) then filtered through a pad of silica. The solvent was removed under vacuum and the residue slurried in petroleum ether (40-60). The product was recovered by filtration. Analytically pure material was obtained by recystallisation from IPA (11.58 g, 72%).

δ_{H} 7.170 (2H, d J=8.0 Hz, Ar—H), 7.131 (2H, d J=8.0 Hz, Ar—H), 7.046 (2H, dd J=8.5 & 1.0 Hz, Ar—H), 6.95-7.00 (6H, m, Ar—H), 6.858 (2H, d J=8.0 Hz, Ar—H), 6.815 (2H, d J=8.0 Hz, Ar—H), 6.779 (1H, tt J=8.0 & 1.0 Hz, Ar—H), 2.037 (3H, s, tolyl-CH₃), 2.002 (3H, s, tolyl-CH₃), 1.374 (9H, s, Si—(CH₃)₃). δ_{C} 153.7, 148.3, 145.64, 145.60, 141.4, 138.2, 135.0, 132.8, 130.2 (Ar—H), 129.45 (Ar—H), 129.41 (Ar—H), 127.3 (Ar—H), 125.3 (Ar—H), 124.0 (Ar—H), 123.5 (Ar—H), 122.5 (Ar—H), 80.1, 28.2 (BOC—(CH₃)₃), 20.74 (tolyl-CH₃), 20.66 (tolyl-CH₃). EI m/z 464.2 (M⁺, 4%), 364.2 (M⁺-BOC, 100%). Elemental analysis: Calculated for C₃₁H₃₂N₂O₂ C, 80.14; H, 6.94; N, 6.03. Found C, 80.38; H, 6.99; N, 6.17.

### Example 23

### 2,11-bis(9,9-dioctyl-9H-fluoren-2-yl)hexabenzo[bc,ef,hi,kl,no,qr]coronene (Compound 8, Scheme 2)

2,11-bis(7-iodo-9,9-dioctyl-9H-fluoren-2-yl)hexabenzo[bc,ef,hi,kl,no,qr]coronene 3 (1 g, 0.64 mmol) was dissolved in dry THF (50 mL) and cooled to −78° C. n-Butyllithium (1 mL, 2.5 M in hexanes) was added dropwise and allowed to stir at −78° C. for 15 min. Water (0.5 mL) was added and the reaction was allowed to warm from −78° C. to 25° C. over 30 min. Solvent was removed and the residual redissolved in CH₂Cl₂ (50 mL) and filtered through a plug of silica. The product was isolated as a yellow powder (0.8 g, 95% yield) after precipitation from MeOH.

m.p. >250° C. UV-vis: λₘₐₓ (thin film)=368 nm. ¹H NMR (500 MHz, 6.25 mM, CDCl₃, 20° C., δ): 0.81 (t, J=7 Hz, 12H, —CH₃), 0.99 (br, 4H, —CH₂—), 1.10 (br, 4H, —CH₂—), 1.24 (br, 40H, —CH₂—), 2.30 (m, 8H, —CH₂—), 7.30 (br t, 4H, HBC-H), 7.55 (m, 6H, fluorene-H), 7.68 (d, J=7 Hz, 2H, fluorene-H), 7.88 (m, 6H, fluorene-H), 7.94 (br d, 4H, HBC-H), 8.14 (br d, 4H, HBC-H), 8.35 (br s, 4H, HBC-H). ¹³C NMR (125 MHz, 75 mM, CDCl₃, 20° C., δ): 151.5, 151.2, 141.1, 140.9, 140.2, 137.0, 128.6, 128.2 (2), 127.1, 127.0, 126.7, 124.5, 123.1, 122.9, 122.1, 121.7, 120.2, 120.0 (2), 119.9, 118.7, 118.1, 118.0, 55.4, 40.6, 31.9, 30.3, 29.5, 29.4, 24.3, 22.7, 14.2. FT-IR (neat, cm⁻¹): 3066, 2953, 2924, 2852, 1617, 1589, 1455, 1380, 1361, 816, 759, 740. MS-MALDI (m/z): M⁺ 1298.59. Elemental analysis: calcd. for C₁₀₀H₉₈, C, 92.40; H, 7.60. found C, 92.5; H, 7.5.

### Example 24

### 2,11-bis(9,9-dioctyl-7-(thiophen-2-yl)-9H-fluoren-2-yl)hexabenzo [bc,ef,hi,kl,no,qr]coronene (Compound 12, Scheme 2)

Compound 3 (200 mg, 0.13 mmol), thiophene-2-boronic acid pinacol ester 9 (64 mg, 0.30 mmol) and Pd(PPh₃)₄ (1 mg) were dissolved in degassed toluene (5 mL). Tetraethylammonium hydroxide solution (20% wt. in water, 2 mL) was thoroughly degassed and added to the reaction mixture. The resulting solution was heated at 90° C. for 14 h and the product was extracted into toluene. The toluene solution was dried over Na₂SO₄ and filtered through a plug of silica. After the removal of toluene, the resulting residue was purified by size exclusion chromatography (Bio-Rad, Bio-Beads S-X1, THF) and a yellow solid (160 mg, 85% yield) was obtained after precipitation from methanol.

UV-vis: λₘₐₓ, nm (ε, cm L mol⁻¹)=367 (1.75×10⁵). ¹H NMR (500 MHz, 7.5 mM, CDCl₃, 20° C., δ): 0.83 (m, 12H, —CH₃), 1.06 (br, 8H, —CH₂—), 1.26 (br, 40H, —CH₂—), 2.30 (m, 8H, —CH₂—), 7.21 (br t, 4H, HBC-H), 7.25 (m, 2H, thiophene-H), 7.43 (m, 2H, thiophene-H), 7.58 (m, 4H, thiophene-H and fluorene-H), 7.73 (d, J=7 Hz, 2H, fluorene-H), 7.79-7.88 (m, 12H, fluorene-H and HBC-H), 8.02 (br s, 4H, HBC-H), 8.22 (br s, 4H, HBC-H). ¹³C NMR (125 MHz, 12 mM, CDCl₃, 20° C., δ): 153.6, 152.0, 151.8, 151.0, 145.4, 140.636, 140.5, 139.2, 133.4, 128.8, 128.4, 128.2, 124.7, 124.6, 123.1, 123.0, 121.7, 121.7, 120.4, 120.3, 120.2, 120.1, 120.1 (3), 118.8, 118.4, 118.2, 92.6, 55.7, 55.5, 40.6, 40.4, 31.9, 30.3, 30.2, 29.6, 29.5 (2), 29.4, 24.3, 22.7 (2), 14.2, 14.1. FT-IR (neat, cm⁻¹): 3070, 2952, 2923, 2852, 1616, 1466, 1456, 1380, 1250, 989, 812, 759, 740, 692. MS-MALDI (m/z): M⁺ 1462.6. Elemental analysis: calcd. for C₁₀₈H₁₀₂S₂, C, 88.60; H, 7.02; S, 4.38. found C, 86.6; H, 7.2. Note: The elemental analysis indicates impurities in the sample and this is thought to be the mono-substituted derivative which can be observed in the MALDI mass spectrum of the sample. Efforts to purify the product by various chromatography techniques including HPLC and recycling GPC were unsuccessful.

### Example 25

### 2,11-bis[9,9-dioctyl-7-(5,5″-bis(trimethylsilyl)-2,2′:3′,2″-terthiophene-5′-yl)-9H-fluoren-2-yl]hexabenzo[bc,ef,hi,kl,no,qr]coronene (Compound 13)

Compound 3 (200 mg, 0.13 mmol), thiophene-2-boronic acid pinacol ester 10 (156 mg, 0.30 mmol) and Pd(PPh₃)₄ (1 mg) were dissolved in degassed toluene (5 mL). Tetraethylammonium hydroxide solution (20% wt. in water, 2 mL) was thoroughly degassed and added to the reaction mixture. The resulting solution was heated at 90° C. for 14 h and the product was extracted into toluene. The toluene solution was dried over Na₂SO₄ and filtered through a plug of silica. After the removal of toluene, the resulting residue was purified by size exclusion chromatography (Bio-Rad, Bio-Beads S-X1, THF) and a yellow solid (230 mg, 86% yield) was obtained after precipitation from methanol.

UV-vis: λₘₐₓ, nm (ε, cm L mol⁻¹)=367 (1.71×10⁵). ¹H NMR (500 MHz, 8 mM, CDCl₃, 20° C., δ): 0.39 (s, 18H, TMS), 0.41 (s, 18H, TMS), 0.82 (m, 12H, —CH₃), 1.06 (br m, 4H, —CH₂—), 1.14 (br m, 4H, —CH₂—), 1.25 (br m, 40H, —CH₂—), 2.36 (m, 8H, —CH₂—), 7.21 (d, J=3 Hz, 2H), 7.24 (d, J=3 Hz, 2H), 7.28 (d, J=3 Hz, 2H), 7.30 (d, J=3 Hz, 2H), 7.60 (s, 4H, ArH), 7.80-7.87 (m, 14H, ArH), 8.01 (br s, 4H, ArH), 8.20 (br s, 4H, ArH). ¹³C NMR (125 MHz, 8 mM, CDCl₃, 20° C., δ): 152.1, 151.8, 143.3, 142.9, 141.8, 141.0, 140.9, 140.7, 140.6, 139.8, 137.2, 134.3, 132.6, 131.0, 128.9, 128.6, 128.5, 128.5, 128.4, 128.0, 126.9, 126.0, 124.9, 123.3, 122.4, 121.7, 120.3, 120.2, 120.2, 118.9, 118.5, 55.6, 40.6, 31.9, 30.3, 29.5 (2), 24.3, 22.7, 14.2, 0.1, −0.0. FT-IR (neat, cm⁻¹): 2928, 2853, 1616, 1468, 1381, 1250, 990, 840, 813, 758. MS-MALDI (m/z): M⁺ 2079.1. Elemental analysis: calcd. for C₁₃₆H₁₄₂S₆Si₄, C, 78.48; H, 6.88; S, 9.24; Si, 5.40. found C, 78.4; H, 6.9; S, 9.41.

### Example 26

### 2,11-bis(9,9-dioctyl-7-(2,2′:3′,2″-terthiophene-5′-yl)-9H-fluoren-2-yl)hexabenzo[bc,ef,hi,kl,no,qr]coronene (Compound 14)

Compound 13 (100 mg, 0.05 mmol) was dissolved in THF (25 mL). Tetrabutylammonium fluoride trihydrate (100 mg, 0.3 mmol) was added and the resulting solution was stirred at 25° C. for 30 min. After the removal of solvent, a yellow solid (80 mg, 89% yield) was obtained after precipitation from methanol.

UV-vis: λₘₐₓ, nm (ε, cm L mol⁻¹)=368 (1.69×10⁵). ¹H NMR (500 MHz, 15 mM, CDCl₃, 20° C., δ): 0.82 (m, 12H, —CH₃), 1.06 (br m, 4H, —CH₂—), 1.13 (br m, 4H, —CH₂—), 1.25 (br m, 40H, —CH₂—), 2.32 (m, 8H, —CH₂—), 7.11 (m, 6H, ArH), 7.23 (m, 4H, ArH), 7.38 (m, 4H, ArH), 7.45 (m, 2H, ArH), 7.59 (s, 2H, ArH), 7.70 (m, 4H, ArH), 7.80 (m, 8H, ArH), 7.90 (br s, 4H, ArH), 8.11 (br s, 4H, ArH). ¹³C NMR (125 MHz, 15 mM, CDCl₃, 20° C., δ): 152.2, 151.8, 143.6, 141.1, 140.9, 139.7, 137.6, 137.1, 135.3, 132.9, 132.5, 131.1, 128.8, 128.4, 127.6, 127.4, 127.3, 126.9, 126.7, 125.9, 125.7, 124.9, 124.8, 123.2, 122.3, 121.7, 120.6, 120.2, 120.1, 118.8, 118.4, 118.3, 55.6, 40.6, 32.0, 30.3, 29.5, 29.5, 24.4, 22.8, 14.2. FT-IR (neat, cm⁻¹): 3069, 2952, 2923, 2851, 1616, 1471, 1380, 813, 759, 740, 694. MS-MALDI (m/z): M⁺ 1790.69. Elemental analysis: calcd. for C₁₂₄H₁₁₀S₆, C, 83.08; H, 6.19; S, 10.73. found C, 83.0; H, 6.3; S, 10.55.

### Example 27

### 2,11-bis[9,9-dioctyl-7-[5,5″″″-bis(trimethylsilyl)-3′,5′″″-bis[5-(trimethylsilyl)-2-thienyl]-2,2′:5′,2″:5″,2′″:3′″,2″″:5″″,2′″″:4′″″,2″″″-septithiophene-5′″-yl]-9H-fluoren-2-yl]hexabenzo[bc,ef,hi,kl,no,qr]coronene (Compound 15)

Compound 3 (100 mg, 0.06 mmol), thiophene-2-boronic acid pinacol ester 11 (173 mg, 0.15 mmol) and Pd(PPh₃)₄ (1 mg) were dissolved in degassed toluene (5 mL). Tetraethylammonium hydroxide solution (20% wt. in water, 2 mL) was thoroughly degassed and added to the reaction mixture. The resulting solution was heated at 90° C. for 14 h and the product was extracted into toluene. The toluene solution was dried over Na₂SO₄ and filtered through a plug of silica. After the removal of toluene, the resulting residue was purified by size exclusion chromatography (Bio-Rad, Bio-Beads S-X1, THF) and a yellow solid (190 mg, 88% yield) was obtained after precipitation from methanol.

UV-vis: λₘₐₓ, nm (ε, cm L mol⁻¹)=367 (2.17×10⁵). ¹H NMR (500 MHz, 2 mM, CDCl₃, 20° C., δ): 0.35 (s, 36H, TMS), 0.36 (s, 18H, TMS), 0.37 (s, 18H, TMS), 0.87 (m, 12H, —CH₃), 1.12 (br m, 8H, —CH₂—), 1.29 (br m, 40H, —CH₂—), 2.32 (m, 8H, —CH₂—), 7.18-7.31 (m, 28H, ArH), 7.61 (s, 2H, ArH), 7.67 (s, 2H, ArH), 7.82-8.00 (m, 18H, ArH), 8.19 (s, 4H, ArH), 8.39 (s, 4H, ArH). ¹³C NMR (125 MHz, 2 mM, CDCl₃, 20° C., δ): 152.3, 151.9, 144.0, 142.4, 142.3, 142.0, 141.2, 140.9, 140.8, 140.1, 139.8, 137.7, 136.9, 136.6, 135.5, 135.2, 134.3, 134.2, 132.3 (2), 130.9, 130.8, 130.7, 129.2, 128.6, 128.6, 128.4, 128.0 (2), 126.7, 126.6, 125.8, 125.0, 124.9, 123.6, 122.7, 121.8, 120.6, 120.4, 120.1, 118.9, 55.6, 40.5, 32.0, 30.3, 29.5, 24.4, 22.7, 14.2, 0.0, −0.1. FT-IR (neat, cm⁻¹): 3056, 2953, 2924, 2852, 1464, 1249, 988, 837, 799, 757. MS-MALDI (m/z): M⁺ 3351.1. Elemental analysis: calcd. for C₁₉₆H₁₉₈S₁₈Si₈, C, 70.16; H, 5.95; S, 17.20; Si, 6.70. found C, 70.2; H, 5.9.

### Example 28

### 2,11-bis[9,9-dioctyl-7-[3′,5′″″-bis(2-thienyl)-2,2′:5′,2″:5″,2′″:3′″,2″″:5″″,2′″″:4′″″,2″″″-septithiophene-5′″-yl]-9H-fluoren-2-yl]hexabenzo[bc,ef,hi,kl,no,qr]coronene (Compound 16)

Compound 15 (100 mg, 0.03 mmol) was dissolved in THF (25 mL). Tetrabutylammonium fluoride trihydrate (100 mg, 0.3 mmol) was added and the resulting solution was stirred at 25° C. for 30 min. After the removal of solvent, a yellow solid (80 mg, 96% yield) was obtained after precipitation from methanol.

UV-vis: λₘₐₓ, nm (ε, cm L mol⁻¹)=369 (1.98×10⁵). ¹H NMR (500 MHz, 8 mM, CDCl₃, 20° C., δ): 0.83 (m, 12H, —CH₃), 1.07 (br m, 4H, —CH₂—), 1.14 (br m, 4H, —CH₂—), 1.26 (br m, 40H, —CH₂—), 2.26 (m, 8H, —CH₂—), 7.01 (m, 6H, ArH), 7.09-7.22 (m, 18H, ArH), 7.30 (m, 6H, ArH), 7.45 (m, 2H, ArH), 7.56 (s, 2H, ArH), 7.65 (m, 2H, ArH), 7.76 (m, 10H, ArH), 7.96 (br s, 4H, ArH), 8.17 (br s, 4H, ArH). ¹³C NMR (125 MHz, 8 mM, CDCl₃, 20° C., δ): 152.3, 151.8, 143.9, 141.1, 140.8, 139.6, 137.5, 137.0, 136.9, 136.8, 136.7, 135.7, 135.9, 134.8, 134.7, 134.4, 132.5, 132.3, 130.9, 130.8, 130.7, 128.9, 128.5, 128.0, 127.8, 127.3, 127.2, 126.9 (2), 126.8, 126.5, 126.4, 125.8 (2), 124.9, 124.4, 123.3, 122.4, 121.7, 120.7, 120.5, 120.1, 118.7, 118.5, 55.5, 40.4 (2), 32.0, 30.4, 30.3, 29.7, 29.6 (2), 29.5, 24.5, 22.7, 14.3, 14.2. FT-IR (neat, cm⁻¹): 2928, 2854, 1465, 1379, 1262, 814, 760, 697. MS-MALDI (m/z): M⁺ 2774.55. Elemental analysis: calcd. for C₁₇₂H₁₃₄S₁₈, C, 74.36; H, 4.86; S, 20.78. found C, 74.4; H, 4.9; S, 18.97.

### Example 29

### Bulk Heterojunction PV Cell Device Fabrication Procedures and Data for HBC-Triarylamine Dendritic Compounds

UV-ozone cleaning was performed using a Novascan PDS-UVT, UV/ozone cleaner with the platform set to maximum height, the intensity of the lamp is greater than 36 mW/cm² at a distance of 100 cm. At ambient conditions the ozone output of the UV cleaner is greater than 50 ppm.

Aqueous solutions of PEDOT/PSS were deposited in air using a Laurell WS-400B-6NPP Lite single wafer spin processor (acceleration=13608 rpm). The active layers were deposited inside a glovebox using an SCS G3P Spincoater (set to maximum acceleration). Film thicknesses were determined using a Dektak 6M Profilometer. Vacuum depositions were carried out using an Edwards 501 evaporator inside a Vacuum Atmospheres argon-filled glovebox (H₂O and O₂ levels both <1 ppm). Samples were placed on a shadow mask in a tray with a source to substrate distance of approximately 25 cm. The area defined by the shadow mask gave device areas of exactly 0.2 cm². Deposition rates and film thicknesses were monitored using a calibrated quartz thickness monitor inside the vacuum chamber. Calcium (Aldrich) and Al (3 pellets of 99.999%, KJ Lesker) were evaporated from open tungsten boats. C₆₀ and C₇₀ PCBM were prepared using literature procedures.⁹

ITO coated glass (Kintek, 15Ω/□) was cleaned by standing in a stirred solution of 5% (v/v) Deconex 12PA detergent at 90° C. for 20 mins. The ITO was then successively sonicated for 10 mins each in distilled water, acetone and iso-propanol. The substrates were then exposed to a UV-ozone clean (at RT) for 10 mins. The PEDOT/PSS (HC Starck, Baytron P A14083) was filtered (0.2 μm RC filter) and deposited by spin coating at 5000 rpm for 60 sec to give a 38 nm layer. The PEDOT/PSS layer was then annealed on a hotplate in the glovebox at 145° C. for 60 mins. Solutions of the polymers were deposited onto the PEDOT/PSS layer by spin coating in the glovebox. The polymers were dissolved in chlorobenzene (Aldrich, anhydrous) in individual vials with stirring. The solutions of P3HT and the block co-polymer were warmed gently to about 80° C. for 1 min to complete the dissolution. All material stayed in solution on cooling to room temperature. The solutions of P3HT and F8BT were then combined, filtered (0.2 μm RC filter) and deposited by spin coating. The solution of the block co-polymer was filtered (0.2 μm RC filter) and deposited by spin coating. Spin speeds were optimised and film thicknesses were measured for each solution. Where noted, the films were then annealed on a hotplate in the glovebox at 140° C. (as measured by a surface thermometer) for 10 min. The devices were transferred (without exposure to air) to a vacuum evaporator in an adjacent glovebox. A layer of Ca (20 nm) and then Al (100 nm) was deposited by thermal evaporation at pressures below 2×10⁻⁶ mbar. A connection point for the ITO electrode was made by manually scratching off a small area of the polymer layers. A small amount of silver paint (Silver Print II, GC electronics, Part no.: 22-023) was then deposited onto all of the connection points, both ITO and Al. The completed devices were then encapsulated with glass and a UV-cured epoxy (Lens Bond type J-91) by exposing to 254 nm UV-light inside a glovebox (H₂O and O₂ levels both <1 ppm) for 10 mins.

The encapsulated devices were then removed from the glovebox and tested in air within 1 hour. Electrical connections were made using alligator clips. The cells were tested with an Oriel solar simulator fitted with a 1000 W Xe lamp filtered to give an output of 100 mW/cm² at AM 1.5. The lamp was calibrated using a standard, filtered Si cell from Peccell Limited. Prior to analysis the output of the lamp was adjusted to give a J_{SC} of 0.605 mA with the standard device. The devices were tested using a Keithley 2400 Sourcemeter controlled by Labview Software.

The Incident Photon Collection Efficiency (IPCE) data was collected using an Oriel 150 W Xe lamp coupled to a monochromator and an optical fibre. The output of the optical fibre was focussed to give a beam that was contained within the area of the device, approximately 1 mm in diameter. The IPCE was calibrated with a standard, unfiltered Si cell.

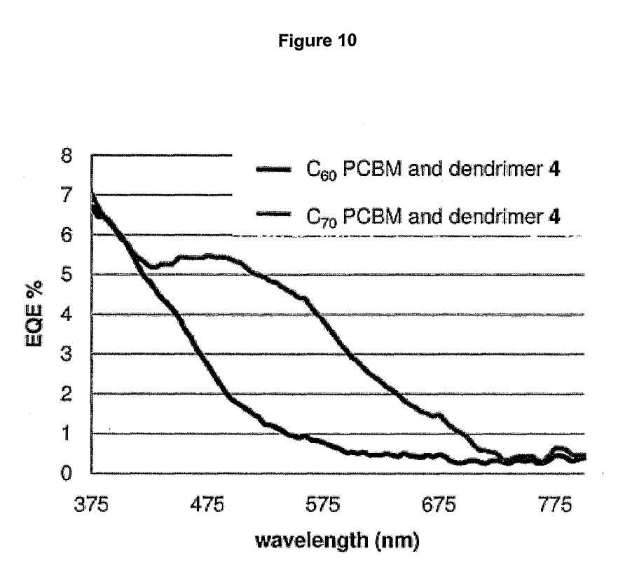

Table 3 shows the experimental details of active layer composition and treatment while Table 4 shows the device data. FIG. 10 shows the EQE spectra of devices with HBC-triarylamine dendrimer 4 and two fullerene derivatives, C₆₀PCBM and C₇₀PCBM. There is a clear contribution from the C₇₀PCBM to photocurrent leading to an increase in power conversion efficiency in the device (0.06% to 0.16%, Table 4).

TABLE 3
Experimental details of active layer composition and
treatment for HBC-triarylamine photovoltaic devices.

| | Materials | | | | | | Spin | Film | Anneal |
|---|---|---|---|---|---|---|---|---|---|
| | 4 | 5 | 6 | C₆₀PCBM | C₇₀PCBM | | speed | thickness | 140° C. |
| Device | (mg) | (mg) | (mg) | (mg) | (mg) | Blend details | rpm | nm | 10 min |
| 1 | 5 | 0 | 0 | 10 | 0 | in 0.6 cm³ | 1500 | 40 | no |
| | | | | | | chlorobenzene | | | |
| 2 | 2.5 | 0 | 0 | 10 | 0 | in 0.5 cm³ | 1500 | 45 | no |
| | | | | | | chlorobenzene | | | |
| 3 | 0 | 5 | 0 | 10 | 0 | in 0.6 cm³ | 1500 | 40 | no |
| | | | | | | chlorobenzene | | | |
| 4 | 0 | 2.5 | 0 | 10 | 0 | in 0.5 cm³ | 1500 | 40 | no |
| | | | | | | chlorobenzene | | | |
| 5 | 0 | 0 | 5 | 11 | 0 | in 0.6 cm³ | 1500 | 50 | no |
| | | | | | | chlorobenzene | | | |
| 6 | 0 | 0 | 2.5 | 11 | 0 | in 0.5 cm³ | 1500 | 42 | no |
| | | | | | | chlorobenzene | | | |
| 7 | 5 | 0 | 0 | 0 | 10 | in 0.6 cm³ | 1500 | 65 | no |
| | | | | | | chlorobenzene | | | |
| 8 | 0 | 5 | 0 | 0 | 10 | in 0.6 cm³ | 1500 | 65 | no |
| | | | | | | chlorobenzene | | | |
| 9 | 0 | 0 | 4.4 | 0 | 8.6 | in 0.5 cm³ | 1500 | 60 | no |
| | | | | | | chlorobenzene | | | |

Device structure is ITO/PEDOT: PSS (30 nm)/active layer (40-60 nm)/Ca (20 nm)/Al (100 nm).

TABLE 4
Table of photovoltaic device data for HBC-triarylamine dendrimers.

| | | | Actual | Light | | | | | | | |
|---|---|---|---|---|---|---|---|---|---|---|---|
| | | Area | Area | power | Voc | | Isc | | Pmax | V at | Power |
| Device | Pixel | input | (cm2) | (W/cm2) | (V) | Isc (A) | (A/cm2) | FF | (W/cm2) | Pmax | Efficiency |
| 1 | 1 | 0.2 | 0.2 | 0.1 | 0.56 | 7.41E−05 | 3.71 E−04 | 0.28 | 5.76E−05 | 3.04E−01 | 0.06 |
| 1 | 2 | 0.2 | 0.2 | 0.1 | 0.49 | 6.75E−05 | 3.37E−04 | 0.26 | 4.33E−05 | 2.44E−01 | 0.04 |
| 1 | 3 | 0.2 | 0.2 | 0.1 | 0.54 | 7.08E−05 | 3.54E−04 | 0.27 | 5.13E−05 | 2.84E−01 | 0.05 |
| 2 | 1 | 0.2 | 0.2 | 0.1 | 0.64 | 1.36E−04 | 6.82E−04 | 0.30 | 1.33E−04 | 3.83E−01 | 0.13 |
| 2 | 2 | 0.2 | 0.2 | 0.1 | 0.52 | 1.38E−04 | 6.88E−04 | 0.27 | 9.74E−05 | 2.64E−01 | 0.10 |
| 2 | 3 | 0.2 | 0.2 | 0.1 | 0.58 | 1.35E−04 | 6.73E−04 | 0.28 | 1.11E−04 | 3.23E−01 | 0.11 |
| 2 | 4 | 0.2 | 0.2 | 0.1 | 0.55 | 1.33E−04 | 6.64E−04 | 0.27 | 9.93E−05 | 2.94E−01 | 0.10 |
| 2 | 5 | 0.2 | 0.2 | 0.1 | 0.50 | 1.34E−04 | 6.68E−04 | 0.29 | 9.63E−05 | 2.74E−01 | 0.10 |
| 3 | 2 | 0.2 | 0.2 | 0.1 | 0.56 | 6.75E−05 | 3.37E−04 | 0.30 | 5.70E−05 | 3.23E−01 | 0.06 |
| 3 | 5 | 0.2 | 0.2 | 0.1 | 0.63 | 6.78E−05 | 3.39E−04 | 0.29 | 6.14E−05 | 3.53E−01 | 0.06 |
| 4 | 1 | 0.2 | 0.2 | 0.1 | 0.63 | 1.38E−04 | 6.90E−04 | 0.30 | 1.31E−04 | 3.73E−01 | 0.13 |
| 4 | 4 | 0.2 | 0.2 | 0.1 | 0.52 | 1.33E−04 | 6.66E−04 | 0.30 | 1.05E−04 | 2.74E−01 | 0.10 |
| 4 | 5 | 0.2 | 0.2 | 0.1 | 0.58 | 1.32E−04 | 6.60E−04 | 0.29 | 1.11E−04 | 3.23E−01 | 0.11 |
| 5 | 1 | 0.2 | 0.2 | 0.1 | 0.46 | 7.11E−05 | 3.55E−04 | 0.27 | 4.40E−05 | 2.44E−01 | 0.04 |
| 5 | 4 | 0.2 | 0.2 | 0.1 | 0.65 | 6.80E−05 | 3.40E−04 | 0.29 | 6.51E−05 | 3.73E−01 | 0.07 |
| 6 | 1 | 0.2 | 0.2 | 0.1 | 0.64 | 1.21E−04 | 6.03E−04 | 0.31 | 1.18E−04 | 3.73E−01 | 0.12 |
| 6 | 2 | 0.2 | 0.2 | 0.1 | 0.51 | 1.25E−04 | 6.27E−04 | 0.27 | 8.79E−05 | 2.74E−01 | 0.09 |
| 6 | 3 | 0.2 | 0.2 | 0.1 | 0.63 | 1.25E−04 | 6.27E−04 | 0.31 | 1.22E−04 | 3.73E−01 | 0.12 |
| 7 | 4 | 0.2 | 0.2 | 0.1 | 0.65 | 1.35E−04 | 6.75E−04 | 0.36 | 1.58E−04 | 4.13E−01 | 0.16 |
| 7 | 5 | 0.2 | 0.2 | 0.1 | 0.49 | 1.30E−04 | 6.52E−04 | 0.30 | 9.77E−05 | 2.54E−01 | 0.10 |
| 8 | 1 | 0.2 | 0.2 | 0.1 | 0.66 | 1.49E−04 | 7.44E−04 | 0.33 | 1.61E−04 | 3.93E−01 | 0.16 |
| 8 | 2 | 0.2 | 0.2 | 0.1 | 0.45 | 1.45E−04 | 7.26E−04 | 0.27 | 8.98E−05 | 2.34E−01 | 0.09 |
| 8 | 3 | 0.2 | 0.2 | 0.1 | 0.65 | 1.42E−04 | 7.11E−04 | 0.33 | 1.51E−04 | 3.83E−01 | 0.15 |
| 9 | 1 | 0.2 | 0.2 | 0.1 | 0.66 | 2.00E−04 | 9.98E−04 | 0.34 | 2.24E−04 | 4.14E−01 | 0.22 |
| 9 | 2 | 0.2 | 0.2 | 0.1 | 0.63 | 2.05E−04 | 1.03E−03 | 0.34 | 2.22E−04 | 3.94E−01 | 0.22 |
| 9 | 3 | 0.2 | 0.2 | 0.1 | 0.64 | 2.01E−04 | 1.00E−03 | 0.34 | 2.18E−04 | 3.94E−01 | 0.22 |
| 9 | 4 | 0.2 | 0.2 | 0.1 | 0.64 | 1.96E−04 | 9.78E−04 | 0.34 | 2.14E−04 | 4.04E−01 | 0.21 |
| 9 | 5 | 0.2 | 0.2 | 0.1 | 0.64 | 1.98E−04 | 9.92E−04 | 0.35 | 2.22E−04 | 4.14E−01 | 0.22 |

Refer to Table 3 for active layer components and device structure.

1. A conjugated compound comprising a conjugated linear or branched polycyclic aromatic or heteroaromatic core, said core being peripherally substituted with at least one conjugated aromatic or heteroaromatic moiety, said moiety or moieties comprising at least one substituent conferring solubility on said compound.

2. The compound of claim 1 wherein said conjugated aromatic or heteroaromatic moiety or moieties modify charge transport mobility within said compound.

3. The compound of claim 1 wherein said conjugated aromatic or heteroaromatic moiety or moieties further comprise at least one terminal substituent located at the conjugation terminus or termini of said moiety or moieties said terminal substituent having reactive functionality.

4. The compound of claim 1 wherein the linear or branched polycyclic aromatic or heteroaromatic core comprises at least three fused or linked aromatic or heteroaromatic rings.

5. The compound of claim 4 wherein the polycyclic aromatic core is hexabenzocoronene.

6. The compound of claim 4 wherein the core is selected from the group comprising porphyrins, confused porphyrins, porphyrazines, and phthalothocyanines.

7. The compound of claim 4 wherein the core contains at least one metal.

8. The compound of claim 1 wherein at least one of the solubility conferring substituents is a branched or unbranched, linear or cyclic, substituted or unsubstituted, hydrocarbyl group.

9. The compound of claim 1 wherein at least one of the solubility conferring substituents confers amphiphilic character on the entire molecule.

10. The compound of claim 8 wherein the solubility conferring substituent is a branched or unbranched, substituted or unsubstituted, linear or cyclic alkyl, alkenyl, or alkynyl group, especially a long chain alkyl, alkenyl or alkynyl group having from between 4 and 30 carbon atoms.

11. The compound of claim 1 wherein at least one of the solubility conferring substituents is laterally bonded to the conjugated aromatic or heteroaromatic moiety or moieties.

12. The compound of claim 1 wherein the substituent having reactive functionality comprises one or more halo, alkenyl, alkynyl, aldehyde, boronic acid, amino, hydroxyl, haloalkyl or carboxylate moieties.

13. The compound of claim 12 wherein the substituent having reactive functionality comprises an iodo moiety.

14. The compound of claim 1 wherein the conjugated aromatic moiety is fluorenyl.

15. A compound or dendrimer formed by the reaction between the reactive functionality located at the conjugated terminus of the conjugated compound of claim 1 and a chain extender.

16. The compound or dendrimer of claim 15 wherein the chain extender is conjugated.

17. The compound or dendrimer of claim 16 wherein the chain extender has electron acceptor or donor characteristics.

18. The compound or dendrimer of claim 17 wherein the chain extender comprises triarylamine or thiophene groups.

19. A heterojunction device comprising as an active component one or more compounds or dendrimers of claim 1.

20. The device of claim 19 further comprising one or more electron donors or acceptors.

21. The device of claim 20 wherein the electron acceptor is a soluble fullerene.

22. The device of claim 21 wherein the fullerene is a C60 or C70 fullerene.

23. A photovoltaic cell comprising a heterojunction device according to claim 19.

24. (canceled)
